(19) United States
(12) Patent Application Publication
(10) Pub. No.: US 2018/0085578 A1
(43) Pub. Date: Mar. 29, 2018

(54) VAGUS NERVE STIMULATION FOR TREATING SPINAL CORD INJURY

(71) Applicant: BOARD OF REGENTS, THE UNIVERSITY OF TEXAS SYTEM, Austin, TX (US)

(72) Inventors: Robert L. RENNAKER, II, Sachse, TX (US); Michael P. KILGARD, Richardson, TX (US); Patrick D. GANZER, Richardson, TX (US); Seth A. HAYS, Richardson, TX (US)

(21) Appl. No.: 15/717,003

(22) Filed: Sep. 27, 2017

Related U.S. Application Data
(60) Provisional application No. 62/400,364, filed on Sep. 27, 2016.

(51) Int. Cl. A61N 1/36

(52) U.S. Cl. CPC A61N 1/36014; A61N 1/36053; A61N 1/36003; A61N 1/36103; A61N 1/36067

(57) ABSTRACT

Provided herein are methods for the treatment of spinal cord injury in a subject by administering vagus nerve stimulation. In particular, the vagus nerve stimulation is administered in combination with conventional rehabilitation training.

### BACKGROUND

This application claims benefit of priority to U.S. Provisional Application Ser. No. 62/400,364, filed Sep. 27, 2016, the entire contents of which are hereby incorporated by reference.

### 1. Field

The present disclosure relates generally to the fields of molecular biology and medicine. More particularly, it concerns methods for the treatment of spinal cord injury.

### 2. Description of Related Art

Spinal cord injury (SCI) reduces independence and quality of life for millions of people worldwide. Tissue damage and tissue loss in SCI are due both to the primary and secondary injury. The latter involves excitotoxicity, increased oxidative stress and increased inflammation. Interventions to limit the extent of secondary injury may greatly improve clinical outcomes. However, there are currently no treatments for this condition, and therefore the prospects of functional recovery are very limited. Intense rehabilitation is the most consistently effective therapy for SCI patients. Nonetheless, serious impairments persist even after years of therapy (Harvey et al., 2009). Preclinical evidence that greater recovery is possible is growing, but clinical translation has been problematic (Dietz and Fouad, 2014). Thus, there is an unmet need for improved methods for the treatment of SCI.

The use of electrical stimulation for treatment of medical conditions is well known. For example, electrical stimulation of the brain with implanted electrodes (i.e., deep brain stimulation) has been approved for use in the treatment of various conditions, including pain and movement disorders such as essential tremor and Parkinson's disease (Perlmutter and Mink, 2006). Another application of electrical stimulation of nerves is the treatment of radiating pain in the lower extremities by stimulating the sacral nerve roots at the bottom of the spinal cord (U.S. Pat. No. 6,871,099).

One particular type of electrical stimulation is vagus nerve stimulation (VNS, also known as vagal nerve stimulation). This technique was developed initially for the treatment of partial onset epilepsy and was subsequently developed for the treatment of depression and other disorders. In this method, the left vagus nerve is ordinarily stimulated at a location within the neck by first implanting an electrode about the vagus nerve during open neck surgery and by then connecting the electrode to an electrical stimulator circuit (e.g., a pulse generator). The pulse generator is ordinarily implanted subcutaneously within a pocket that is created at some distance from the electrode, which is usually in the left infraclavicular region of the chest. A lead is then tunneled subcutaneously to connect the electrode assembly and pulse generator. The patient's stimulation protocol is then programmed using a device (a programmer) that communicates with the pulse generator, with the objective of selecting electrical stimulation parameters that best treat the patient's condition (e.g., pulse frequency, stimulation amplitude, pulse width). While vagus nerve stimulation is used for the treatment of certain types of intractable epilepsy and treatment-resistant depression, its potential for use in the treatment of other diseases or disorders is unknown.

### SUMMARY

Accordingly, the present disclosure provides methods of treating spinal cord injury (SCI) using vagus nerve stimulation (VNS). In one embodiment, there is provided a method of treating a spinal cord injury in a subject comprising applying an electrical signal to a vagus nerve of said subject. In some aspects, the electrical signal is monophasic, biphasic, or a combination thereof. In certain aspects, the vagus nerve is further defined as the left vagus nerve or the right vagus nerve. In particular aspects, the subject is human.

In some aspects, treating results in increased in neural plasticity, increased motor circuit connectivity, improved motor function, improved sensory function, enhanced voluntary motor control, and/or prevention of secondary injury. In particular aspects, treating results in at least a 50% improvement in motor function.

In some aspects, the electrical signal is administered 1 day to 1 year, or 1 day to 10 years after the spinal cord injury. In certain aspects, the electrical signal is administered in combination with rehabilitation. In some aspects, the electrical signal is administered simultaneously with rehabilitation. In particular aspects, the rehabilitation comprises physical therapy.

In certain aspects, the spinal cord injury is at one or more of the cervical vertebrae, thoracic vertebrae, lumbar vertebrae, or sacral vertebrae. In certain aspects, the spinal cord injury is caused by contusion of the spinal cord, bruising of the spinal cord, loss of blood to the spinal cord, pressure on the spinal cord, cut spinal cord, or severed spinal cord. In some aspects, the spinal cord injury is the result of a physical trauma, infection, insufficient blood flow, or a tumor. In certain aspects, the spinal cord injury is complete spinal cord injury or incomplete spinal cord injury. In some aspects, the incomplete spinal cord injury is anterior cord syndrome, central cord syndrome, Brown-Sequard syndrome, injuries to individual nerve cells or spinal contusion.

In some aspects, applying is further defined as transmitting said electrical signal transcutaneously to the subject to generate an electrical impulse at or near the vagus nerve fibers. In particular aspects, transmitting transcutaneously is effected using a device with an electrically permeable surface for transmitting said electrical signal through the skin of said subject. In some aspects, the device further comprises a signal generator, and one or more electrodes coupled to the signal generator. In specific aspects, the vagus nerve fibers are at least 0.5 cm to 2 cm below the skin of said subject. In some aspects, transmitting subcutaneously is effected using a surgically implanted electrode.

In certain aspects, the electrical signal comprises bursts of pulses with a frequency of 1 to 100 bursts per second. In some aspects, each burst contains 1 to 30 pulses. In particular aspects, each burst contains 10 to 20 pulses, such as 10, 11, 12, 13, 14, 15, 16, 17, 18, 19, 20 or more pulses. In some aspects, the pulses are full sinusoidal waves or square waves. In certain aspects, each burst has a wave frequency of 1 to 100 Hz. In some aspects, each burst has a wave frequency of 25 to 40 Hz, such as 30 Hz. In particular aspects, each pulse is 10 to 1000 microseconds in duration. In some aspects, each pulse is 50 to 200 microseconds in duration. In particular aspects, each pulse is 75 to 150 microseconds in duration. In certain aspects, the electrical signal has a current of 0.1 to 2.0 mA. In some aspects, the electrical signal has a current of 0.5 to 1.0 mA. In certain aspects, the electrical signal has a duration of 100 to 1000 milliseconds. In some aspects, the electrical signal has a duration of 250 to 750 milliseconds. In certain aspects, the electrical signal is applied one to 150 times, or even one to 500 times during a therapy session.

In some aspects, the electrical impulses generate an electric field at the vagus nerve above a threshold for generating action potentials within fibers of the vagus nerve responsible for activating neural pathways, thereby causing release of neurotransmitters within a brain of the patient.

In additional aspects, the method further comprises administering at least one additional therapy. In some aspects, the at least one additional therapy comprises administering a stem cell, one or more growth factors, one or more hormones, and/or a tissue graft. In particular aspects, the tissue graft is a nerve graft. In specific aspects, the stem cell is a neuroprogenitor cell, embryonic stem cell, neural stem cell, mesenchymal stromal cell, Schwann cells, neuron, induced pluripotent stem cell, or a combination thereof. In some aspects, the growth factor is brain derived neurotrophic factor (BDNF), neurotrophin-3 (NT-3), acidic fibroblast growth factor (aFGF; FGF-1), hepatocyte growth factor (HGF), or a combination thereof.

In some aspects, the method further comprises monitoring motor function and/or sensory function in the subject. In particular aspects, monitoring comprises performing an MRI, Diffusion Tensor Imaging (DTT), EMG, PET scan, or SPECT scan.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description It is contemplated that any method or composition described herein can be implemented with respect to any other method or composition described herein,

Throughout this application, the term “about” is used to indicate that a value includes the inherent variation of error for the device, the method being employed to determine the value, or the variation that exists among the study subjects.

Unless it is otherwise clear that a single entity is intended, terms such as “a,” “an,” and “the” are not intended to refer to only a singular entity and include the general class of which a specific example is described for illustration.

In addition, unless it is clear that a precise value is intended, numbers recited herein should be interpreted to include variations above and below that number that may achieve substantially the same results as that number, or variations that are “about” the same number.

Finally, a derivative of the present disclosure may include a chemically modified molecule that has an addition, removal, or substitution of a chemical moiety of the parent molecule.

The following drawings form part of the present specification and are included to further demonstrate certain aspects of the present disclosure. The disclosure may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

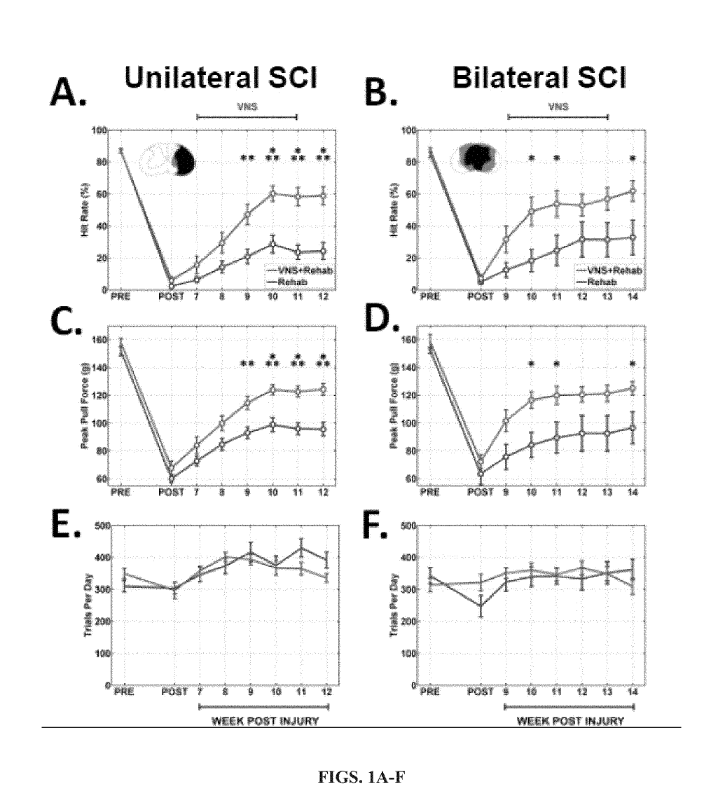

FIGS. 1A-F: VNS paired with rehabilitative training improved forelimb recovery after cervical SCI. (FIG. 1A) VNS paired with rehabilitative training (N=14) improved hit rate (>120 g pulls) compared to rehabilitative training without VNS (N=17) in rats with unilateral SCI. Recovery was maintained after the cessation of VNS on week eleven, which demonstrates that the benefits of VNS are long-lasting. (FIG. 1B) VNS paired with rehabilitative training (N=9) improved hit rate compared to rehabilitative training without VNS (N=9) in rats with bilateral SCI. Recovery was maintained after the cessation of VNS on week thirteen. (FIGS. 1C-D) VNS paired with rehabilitative training improved force production compared to rehabilitative training without VNS in rats with unilateral SCI (FIG. 1C) and bilateral SCI (FIG. 1D). (FIGS. 1E-F). The number of trials performed by the Rehab and VNS+Rehab groups were not different at any time point during testing, which suggest that VNS does not alter motivation. The VNS+Rehab rats received approximately two hundred half-second bursts of VNS per day, which represents less than one percent of the VNS charge delivery approved by the FDA for epilepsy. Significant group differences based on a two-way repeated measures ANOVA with Tukey post-hoc tests are indicated by * for P<0.05, ** for P<0.01, and *** for P<0.005. Significant reductions compared to pre-lesion performance based on a two-way repeated measures ANOVA with Tukey post-hoc tests are indicated by open symbols (P<0.05). These behavioral results demonstrate that pairing VNS with rehabilitation increased motor recovery compared to rehabilitation alone.

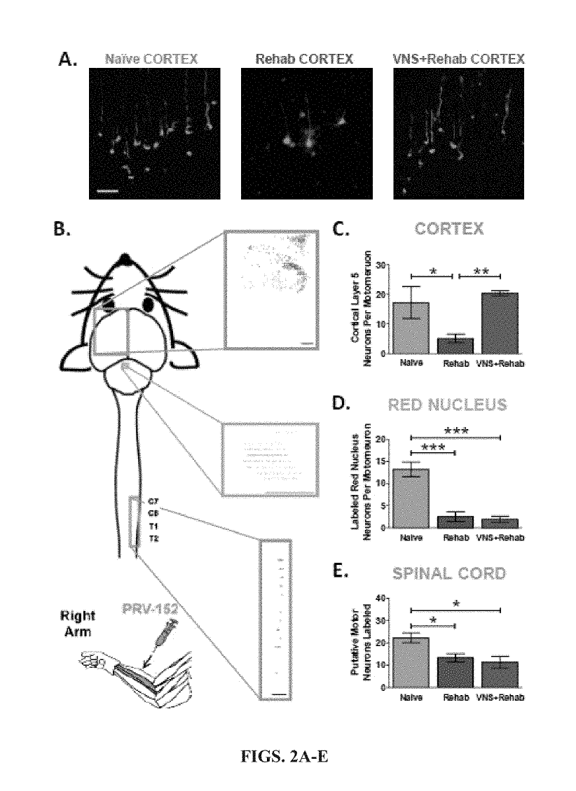

FIGS. 2A-E: VNS paired with rehabilitation increased anatomical connection from the cortex to the grasping muscles of the forelimb compared to rehabilitation alone. (FIG. 2A) Representative photomicrographs of layer 5 sensorimotor cortical labeling are shown for each group (scale bar=100 μm). Pictures are taken from coronal sections through forelimb sensorimotor cortex contralateral to the injected arm. (FIG. 2B) Pseudorabies virus (PRV-152) was injected into the grasping muscles leading to trans-synaptic retrograde labeling of spinal motor, red nucleus and cortical layer 5 neurons (grasping muscles=flexor digitorum profundus and palmaris longus). PRV-152 causes expression of enhanced green florescent protein to label synaptically, connected neurons. Cell locations from a naive animal are plotted as green points within the spinal cord (bottom), red nucleus (middle) and cortex (top) regions of interest (scale bars=1 mm; RFA=rostral forelimb area; CFA=caudal forelimb area). (FIG. 2C) VNS paired with rehabilitation increased the number of labeled cortical layer 5 neurons per labeled spinal motor neuron compared to rehabilitation alone. SCI reduced the number of contralesional red nucleus neurons (FIG. 2D) and ipsilesional spinal motor neurons (FIG. 2E). VNS did not alter the number of red nucleus or spinal motor neurons labeled. Significant differences are determined from one-way ANOVAs with Tukey post-hoc tests (Naïve, N=5; Rehab, N=6; VNS+Rehab, N=5). Differences are indicated by one asterisk for P<0.05, two asterisks for P<0.01 and three asterisks for P<0.001. Collectively, these anatomical results demonstrate that pairing VNS with rehabilitation increased cortical neural plasticity compared to rehabilitation alone. Note that the spinal motor neuron pools for the flexor digitorum profundus and palmaris longus are located from C7 to T2, which is below the level of the SCI.

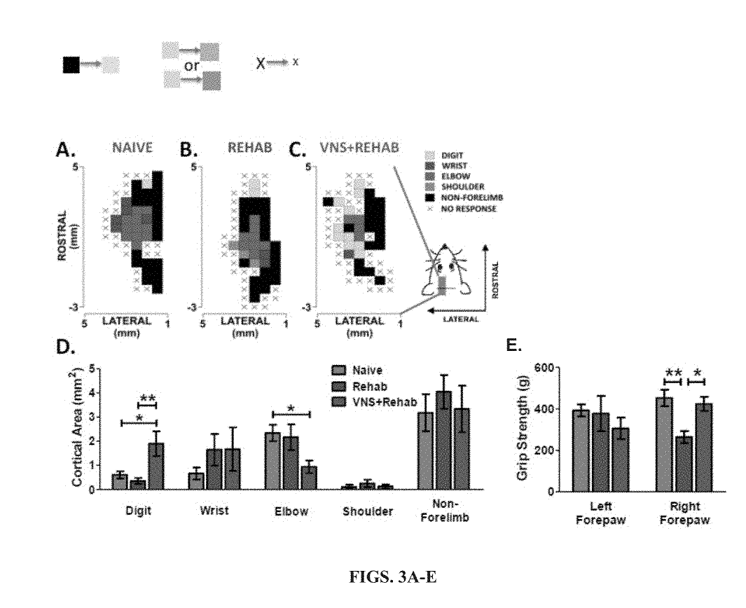

FIGS. 3A-E: VNS paired with rehabilitation increased functional connection from the cortex to the grasping muscles of the forelimb compared to rehabilitation alone. (FIGS. 3A-C) Representative maps of motor cortex derived from intracortical microstimulation studies. X and Y axis coordinates on maps are relative to bregma (mm). Mapping occurred in the left cortex contralateral to the SCI. (FIG. 3D) Pairing VNS with rehabilitation more than doubled the number of motor cortex locations that generate grasping movements of the digits (Naive, N=7; Rehab, N=6; VNS+Rehab, N=6), Significant differences determined from a two-way ANOVA followed by Tukey post-hoc tests. Differences are indicated by one asterisk for P<0.05 and two asterisks for P<0.01. These physiological results demonstrate that pairing VNS with rehabilitation increased neural plasticity compared to rehabilitation alone. (FIG. 3E) VNS paired with rehabilitative training improved the trained limb grip strength compared to rehabilitative training without VNS in rats with unilateral SCI (Naive, N=7; Rehab, N=6; VNS±Rehab, N=7). Grip strength was collected twelve weeks after unilateral SCI. These behavioral results demonstrate that the benefit of pairing VNS with rehabilitation can generalize to another task.

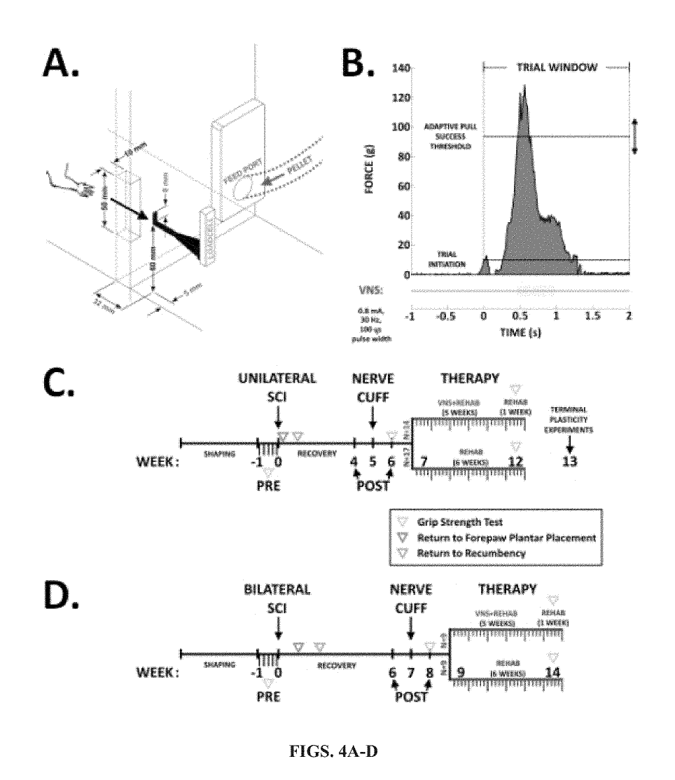

FIGS. 4A-D: Schematic illustration of the behavioral apparatus, VNS delivery timing, and experimental timeline. (FIG. 1A) Prior to SCI, rats were trained to reach through a narrow slit, grasp, and pull a handle with 120 grams of force to receive a food reward. The force profile for every pull trial was measured with a load cell and recorded using custom software. (FIG. 4B) Schematic of a pull trial from a VNS+Rehab animal. A pull trail initiates after 10 g of pull force is measured on the handle. A food pellet reward is delivered if pull force crosses a threshold within 2 seconds of trial initiation. After SCI, rewards were delivered on every trial that exceeded 120 grams (fixed 120 g threshold) or the median peak pull force for the last ten trials (adaptive threshold). This adaptive threshold design ensured that SCI rats received sufficient rewards to stay engaged with the task and were required to perform a challenging motor task during rehabilitation. For rats in the VNS+Rehab group, a 0.5 second burst of VNS was also delivered on each successful trial. The fifteen biphasic pulses (100 us phase) were delivered to the left cervical vagus nerve at 30 Hz and 0.8 mA. Previous studies have shown that left VNS bilaterally activates the target nucleus (nucleus tractus solitarius) while avoiding activation of the sinoatrial node. (FIGS. 4C-D) Experimental timeline containing surgical and behavioral assessment time points for the unilateral SCI (FIG. 4C) and bilateral SCI (FIG. 4D) studies. Therapy lasted 6 weeks for each study and started at week 7 for unilateral SCI and at week 9 for bilateral SCI. Bilateral cervical SCI rats were given two more weeks of recovery time because they required more time to return to sternal recumbency (2^{nd} triangle after SCI) and right forepaw plantar placement (1^{st} triangle after SCI) compared to rats with unilateral cervical SCI. Each therapy week consisted of 5 training days (two 30 minute training sessions per day). To quantify the effect of the adaptive threshold rehabilitation procedure and to ensure that rats were motivated to pull as hard as they could, every fifth day rats were required to perform the fixed 120 g threshold task that they had trained on prior to SCI (longer vertical lines). Fixed 120 g threshold training days are indicated by vertical lines. Light triangles indicate the days that grip strength was tested. VNS-Rehab indicates the period during Which VNS was paired with rehabilitation.

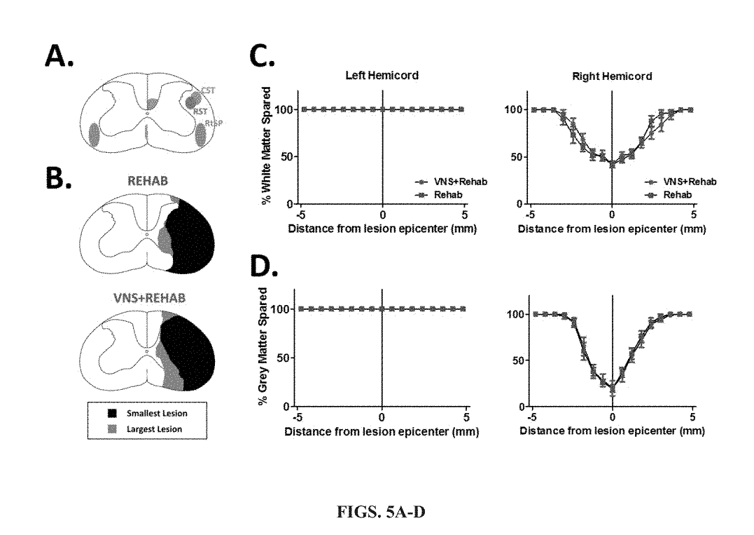

FIGS. 5A-D: Quantification of grey and white matter damage following unilateral cervical spinal cord injury (SCI). (FIG. 5A) Schematic diagram showing the location of the spinal motor neurons in the spinal grey matter and the location of the corticospinal (CST), rubrospinal (RST) and reticulospinal (RtSP) tracts in the spinal white matter. (FIG. 5B) The minimal and maximal lesion extent is shown for all unilateral SCI rats in the Rehab only group (square) and the VNS+Rehab group (circle). (FIGS. 5C-D) VNS did not alter the extent of SCI, which suggests that VNS did not improve motor performance (FIG. 1) by reducing lesion severity. Both groups had extensive damage to the spinal white matter (FIG. 5C) and spinal grey matter (FIG. 5D) that was limited to the right side. The lesion was generated using the Infinite Horizon Impact Device at a force of 200 kilodynes. The impact was delivered at C5/C6, because cervical spinal cord is the most common injury site in patients.

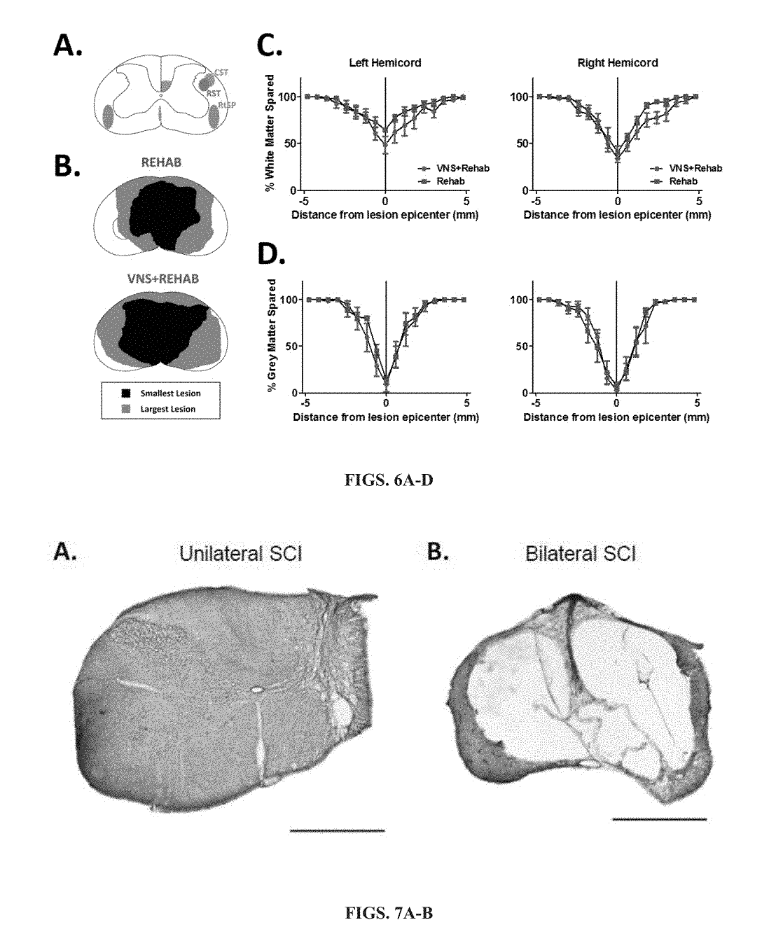

FIGS. 6A-D: Quantification of grey and white matter damage following bilateral cervical spinal cord injury (SCI). (FIG. CA) Schematic diagram showing the location of the spinal motor neurons in the spinal grey matter and the location of the corticospinal (CST), rubrospinal (RST) and reticulospinal (RtSP) tracts in the spinal white matter (FIG. 6B). The minimal and maximal lesion extent is shown for all bilateral SCI rats in the Rehab only group (square) and the VNS+Rehab group (circle). (FIGS. 6C-D) VNS did not alter the extent of SCI, which suggests that VNS did not improve motor performance (FIG. 1) by reducing lesion severity. Both groups had extensive damage to the spinal white matter (FIG. 6C) and spinal grey matter (FIG. 6D) on the right and left sides. The lesions were generated using the Infinite Horizon Impact Device at a force of 200 kilodynes. The midline impact was delivered at C5/C6, because bilateral cervical spinal cord injury is the most common form of SCI.

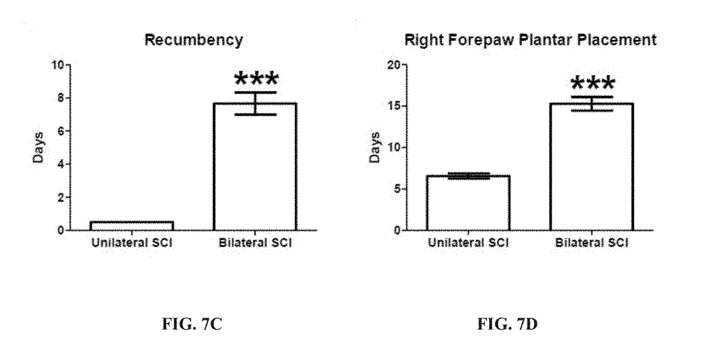

FIGS. 7A-D: Unilateral and bilateral spinal cord injury (SCI) histology. (FIG. 7A) Representative coronal section at C6 in a rat with unilateral SCI (largest hemicontusion). This rat received VNS paired with rehabilitation and had an average hit rate of 74.5% on week 12. (FIG. 7B) Coronal section at C6 in a rat with bilateral SCI (largest midline contusion). This rat received VNS paired with rehabilitation and had an average hit rate of 74.6% on week 14. Bilateral cervical SCI rats (N=16) required more time to return to recumbency (FIG. 7C) and right forepaw plantar placement (FIG. 7D) compared to rats with unilateral cervical SCI (N=31). These functional results indicate that the bilateral SCI was a more severe injury than the unilateral SCI. Rats in the bilateral SCI group were given more time to recover before beginning rehabilitative training. Results are from independent samples t-tests. Differences are indicated by three asterisks for P<0.001.

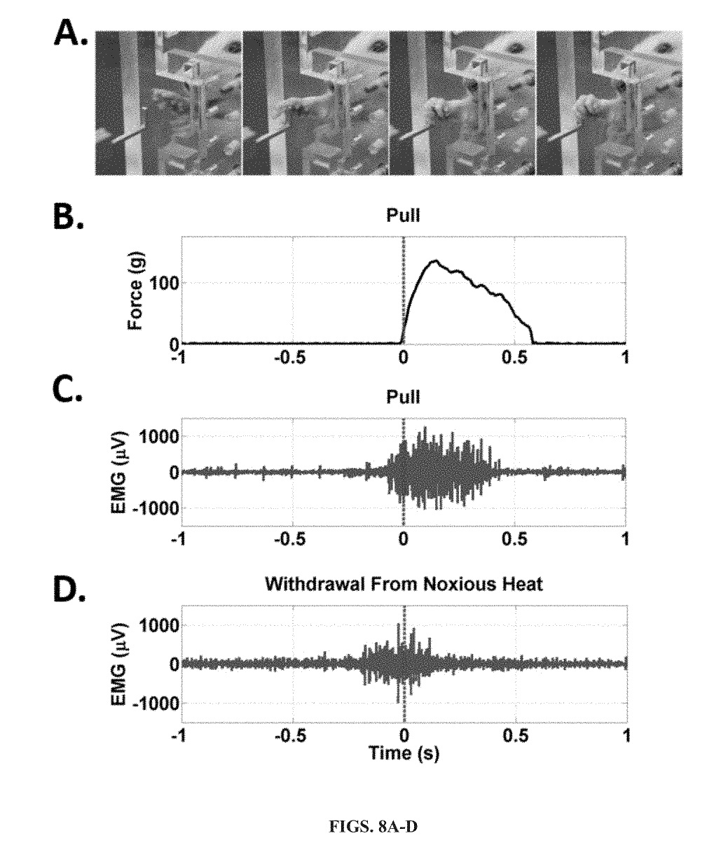

FIGS. 8A-D: Illustration of the EMG data collected during the isometric pull task (FIGS. 8A-C) and the withdrawal from noxious heat (FIG. 8D). (FIG. 8A) Photographs illustrate a typical reach-grasp-pull sequence. (FIG. 8B) Each trial generates a force time series that is used to determine pellet delivery and VNS delivery. A trial is initiated when the force exceeds 10 g (time zero). (FIG. 8C) Biceps EMG activity was recorded for every trial. (FIG. 8D) A Hargreaves device was used to slowly heat the paw from below until the rat withdrew the paw from the heat source (time zero). Biceps EMG precedes both volitional and reflexive movement of the forepaw and was used to evaluate muscle function and hyperreflexia before and after SCI.

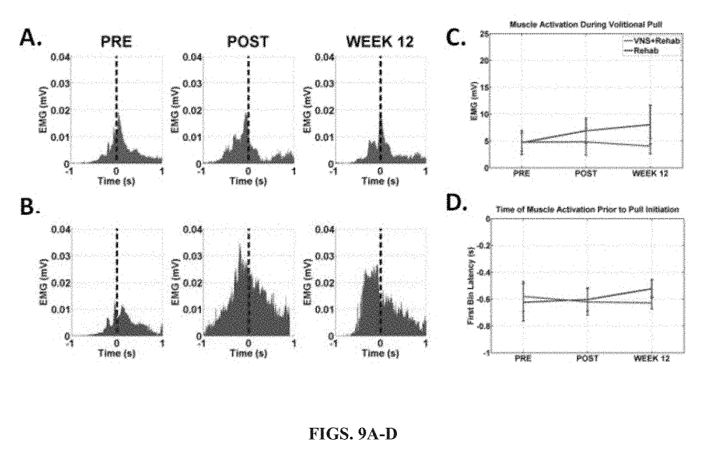

FIGS. 9A-D: Biceps EMG activity during the isometric pull task was not significantly different between VNS+Rehab and Rehab alone. (FIG. 9A) Average EMG activity 1 second before and after pull trial initiation for the VNS+Rehab group (N=4; pull trial initiation=vertical black dashed line at time 0), (FIG. 9B) Average EMG activity 1 second before and after pull trial initiation for the Rehab group (N=4; pull trial initiation=vertical black dashed line at time 0). (FIG. 9C) EMG activity was quantified from the linear envelope of the rectified voltage 1 second before and after each pull trial initiation across animals and time. There were no significant differences in EMG activation magnitude across group or time (F[2,14]=0.5, P=0.582). (FIG. 9D) The first bin latency of EMG activation was calculated as the first EMG time point crossing a 95% confidence interval. The timing of the EMG response relative to pull initiation was also not different across time or group. The finding that EMG activity was not significantly different between VNS+Rehab rats and Rehab alone rats suggests that VNS did not improve forelimb motor performance by increasing elbow flexor muscle activation or reducing muscle atrophy.

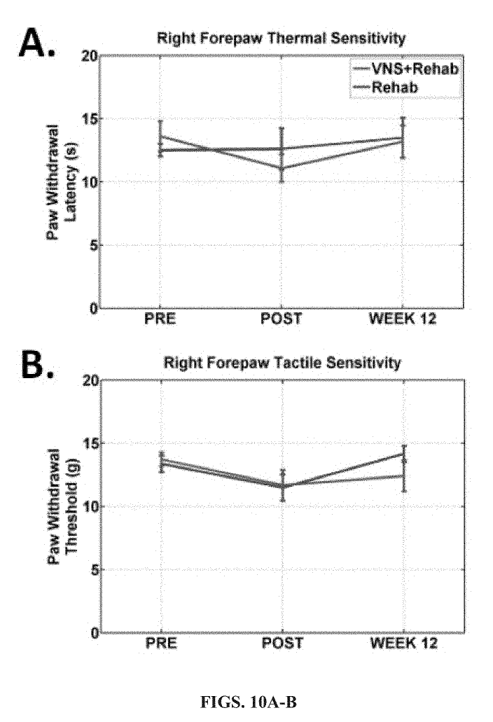

FIGS. 10A-B: Sensory withdrawal thresholds were not significantly different between VNS+Rehab and Rehab alone. (FIG. 10A) The sensitivity to thermal stimulation was quantified as the time to paw withdrawal after initiation of paw heating using a Hargreaves device (VNS+Rehab, N=7; Rehab, N=7). There were no significant differences across group or time (F[2,18]=0.3, P=0.745). (FIG. 10B) Tactile sensitivity was quantified as the number of grams produced by von Frey filaments that triggered paw withdrawal (VNS+Rehab, N=11; Rehab, N=14). There were no significant differences across group or time (F[2,44]=2.3, P=0.107). The sensory withdrawal thresholds reported are for the right forepaw. The observation that withdrawal thresholds were not significantly different between VNS+Rehab and Rehab alone suggests that VNS did not improve forelimb motor performance (FIG. 1) by reducing pain.

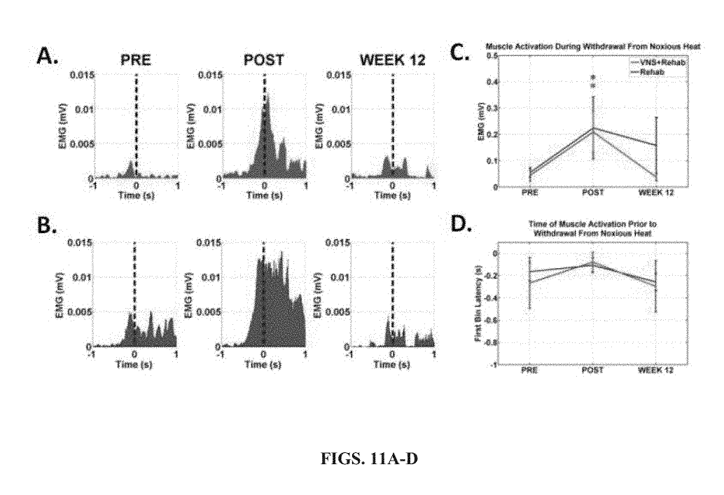

FIGS. 11A-D: Biceps EMG activity during withdrawal from noxious heat was not significantly different between VNS+Rehab and Rehab alone. (FIG. 11A) Average EMG activity 1 second before and after limb withdrawal for the VNS+Rehab group (N=4; limb withdrawal initiation=vertical black dashed line at time 0). (FIG. 11B) Average EMG activity 1 second before and after pull trial initiation for the Rehab group (N=5; limb withdrawal initiation=vertical black dashed line at time 0). (FIG. 11C) EMG activity was quantified from the linear envelope of the rectified voltage 1 second before and after each limb withdrawal initiation across animals and time. (FIG. 11C) As expected from in earlier studies, SCI significantly increased the EMG activity generated by withdrawal from noxious heat for both groups (POST). Significant increases compared to pre-lesion activity (PRE) based on a two-way repeated measures ANOVA followed by simple contrasts. Differences compared to PRE are indicated by asterisks (P<0.05). There were no significant differences between groups at any time point. (FIG. 11D). The first bin latency of EMG activation was calculated as the first EMG time point crossing a 95% confidence interval. The timing of the EMG response relative to limb withdrawal was also not different between groups. The finding that EMG activity was not significantly different between VNS+Rehab rats and Rehab alone rats suggests that VNS did not improve forelimb motor performance (FIG. 1) by reducing hyperreflexia.

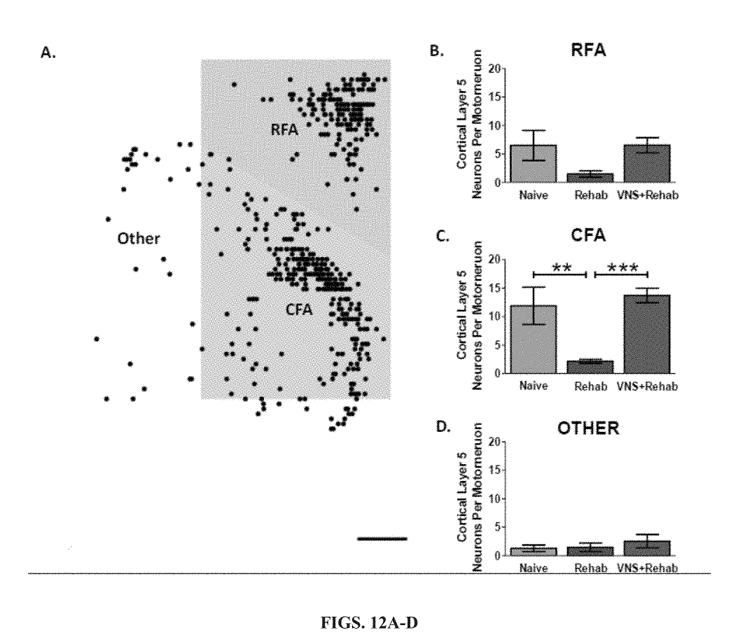

FIGS, 12A-D: VNS paired with rehabilitation increased the anatomical connection from the caudal forelimb area to the grasping muscles of the forelimb compared to rehabilitation alone. (FIG. 12A) Sensorimotor cortex was divided into the rostral forelimb area (RFA, top right shaded region), caudal forelimb area (CFA, lower right shaded region) and OTHER (white left region) regions of interest. Cell locations from a naive animal are plotted as black points within RFA, CFA and OTHER (scale bar=1 mm). VNS paired with rehabilitation significantly increased the number of labeled cortical layer 5 neurons per labeled spinal motor neuron compared to rehabilitation alone in the CFA (FIG. 12C) but not RFA (FIG. 12B) or OTHER (FIG. 12D). Significant differences are determined from one-way ANOVAs with Tukey post-hoc tests (Naïve, N=5; Rehab; N=6; VNS+Rehab, N=5). Differences are indicated by two asterisks for P<0.01 and three asterisks for P<0.001.

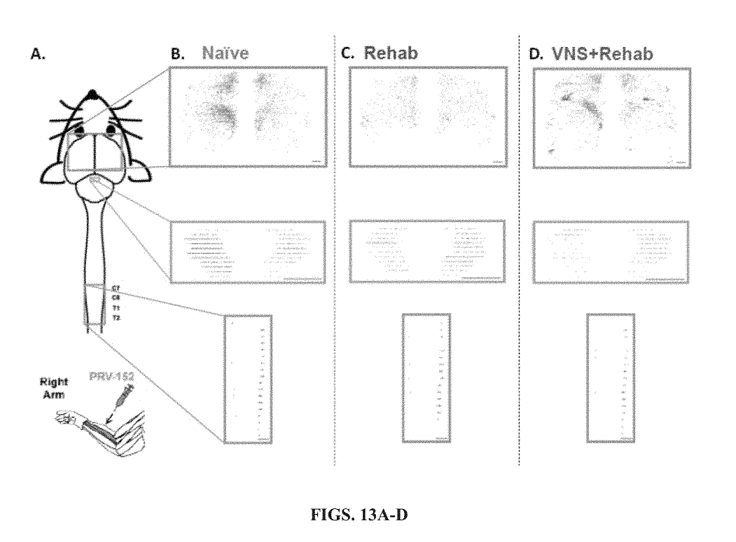

FIGS. 13A-D: Topography of PRV labeled neurons in the spinal cord, red nucleus and sensorimotor cortex. (FIG. 13A) Pseudorabies virus (PRV-152) was injected into the grasping muscles leading to trans-synaptic retrograde labeling of spinal motor, red nucleus and cortical layer 5 neurons (grasping muscles=flexor digitorum profundus and palmaris longus). PRV-152 causes expression of enhanced green florescent protein to label synaptically connected neurons. The locations of labeled neurons are plotted as black points within the spinal cord (bottom), red nucleus (middle) and cortex (top) regions of interest for all Naive (FIG. 13B: n=5), Rehab (FIG. 13C: n=6) and VNS+Rehab (FIG. 13D: n=5) animals. Scale bars in the bottom right of each panel are 1 mm long.

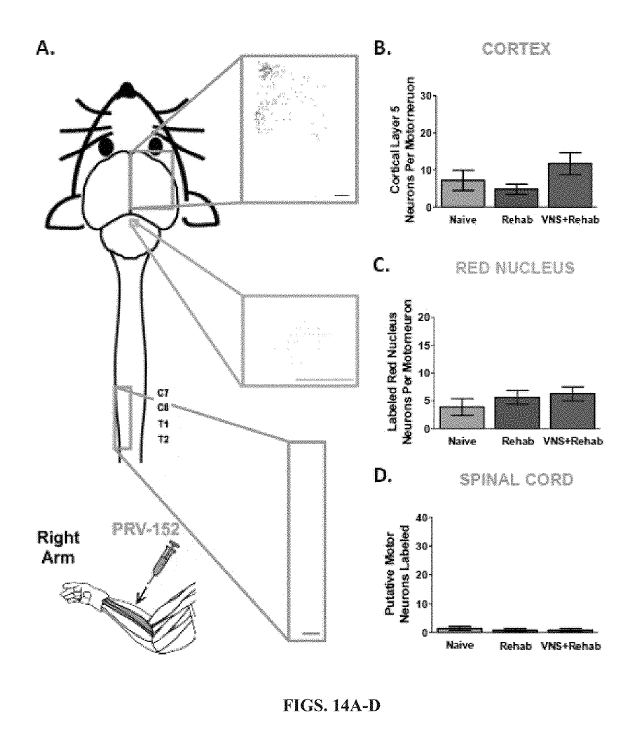

FIGS. 14A-D: VNS paired with rehabilitation did not significantly alter anatomical connectivity in the ipsilesional cortex, ipsilesional red nucleus or contralesional spinal cord. Pseudorabies virus (PRV-152) was injected into the grasping muscles leading to trans-synaptic retrograde labeling of spinal motor, red nucleus and cortical layer 5 neurons (grasping muscles=flexor digitorum profundus and palmaris longus). PRV-152 causes expression of enhanced green florescent protein to label synaptically connected neurons. (FIGS. 14A-C) Schematic of cortex, red nucleus and spinal cord in the left column. No significant differences were identified for neuron counts in the ipsilesional cortex (FIG. 14A), ipsilesional red nucleus (FIG. 14B) or the contralesional spinal cord (FIG. 14C) using one-way ANOVAs (A & C: Naïve, N=5; Rehab, N=6; VNS+Rehab, N=5; B: Naïve, N=3; Rehab, N=3; VNS+Rehab, N=3). These results suggest that VNS did not generate anatomical plasticity on the untrained side of the spinal cord, red nucleus or cortex.

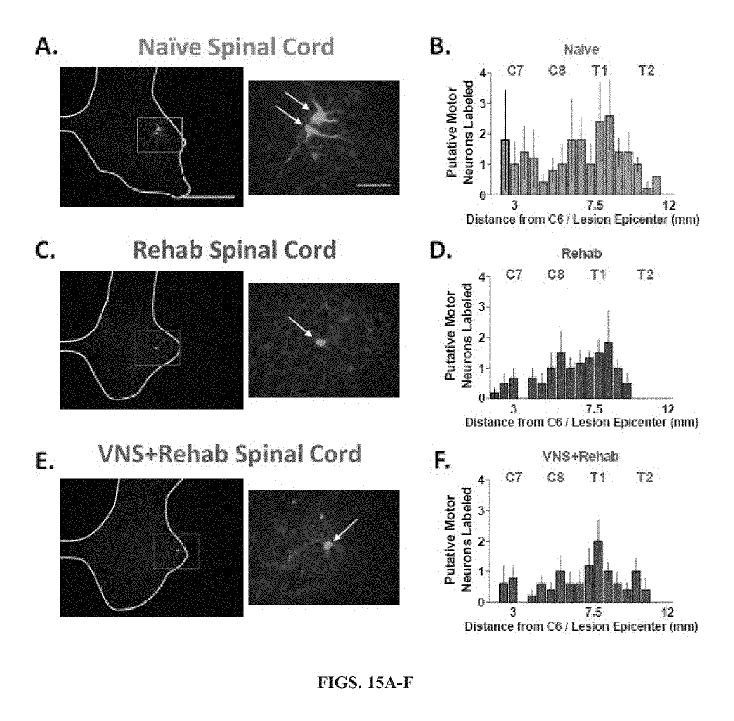

FIGS. 15A-F: SCI reduced the number of spinal motor neurons labeled after pseudorabies virus (PRV-152) was injected into the grasping muscles of the forelimb. (FIGS. 15A, C, F) Representative coronal sections of the right hemicord from each group are shown along with high magnification images of labeled spinal motor neurons labeled from the grasping muscles (flexor digitorum profundus and palmaris longus). The scale bar is 500 μm for the images in the left column and 100 μm for the images in the right column. Spinal gray matter is outlined in white. (FIGS. 15B, D, F) Distribution of spinal motor neurons from C7 to T2 for Naive (N=5), Rehab (N=6) and VNS+Rehab (N=5). Relative spinal levels are shown across the top of B, D and F. Spinal motor neuron counts are binned every 600 μm. No spinal motor neurons were observed above C7 or below T2.

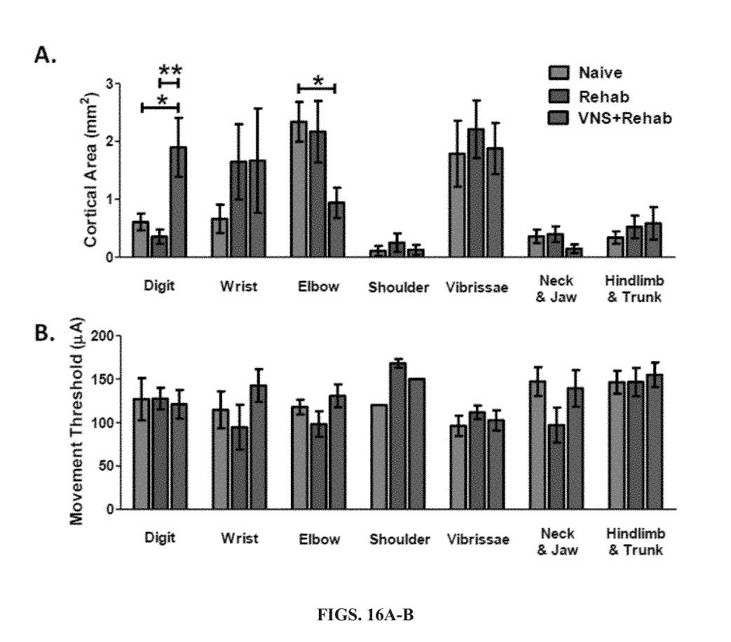

FIGS. 16A-B: Non-forelimb cortical area and movement thresholds for intracortical microstimulation studies. (FIG. 16A) VNS+Rehab or Rehab did not alter the cortical area for any non-forelimb movement (Non-forelimb in FIG. 3D; (Naive, N=7; Rehab, N=6; VNS+Rehab, N=6). (FIG. 16B) There was no significant difference in the current needed to elicit movements during intracortical mapping across groups. Significant differences based on two-way ANOVAs followed by Tukey post-hoc tests. Differences are indicated by one asterisk for P<0.05 and two asterisks for P<0.01.

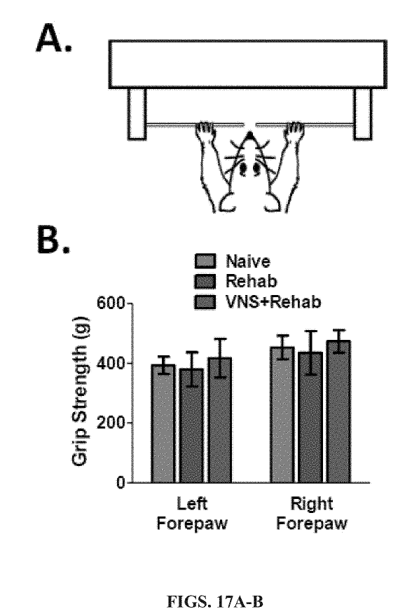

FIGS. 17A-B: Bilateral SCI rats did not exhibit impaired grip strength. (FIG. 17A) Rats gripped two separate bars with each forepaw while being pulled away to measure forepaw gripping strength. (FIG. 17B) Rats with bilateral SCI failed to exhibit an impairment in grip strength (Naïve, N=7; Rehab, N=5; VNS+Rehab, N=5). Grip strength was collected fourteen weeks after bilateral SCI. There were no significant differences between bilateral rats that received VNS and those that did not. Significant group differences based on two-way ANOVAs followed by Tukey post-hoc tests.

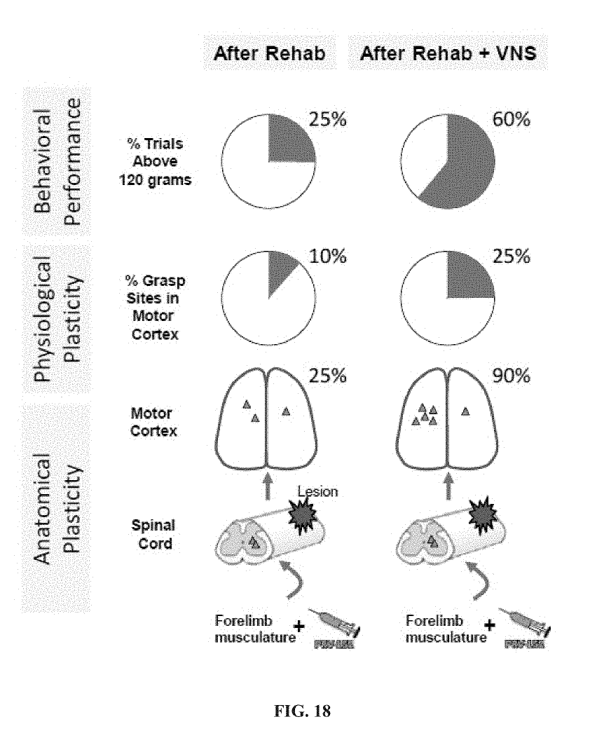

FIG. 18: Graphical summary of the anatomical, physiological, and behavioral benefits of adding VNS to rehabilitation. Percentages indicate the proportion of successful trials, the proportion of motor cortex sites that close the digits, and the proportion of labeled motor cortex neurons compared to unlesioned rats.

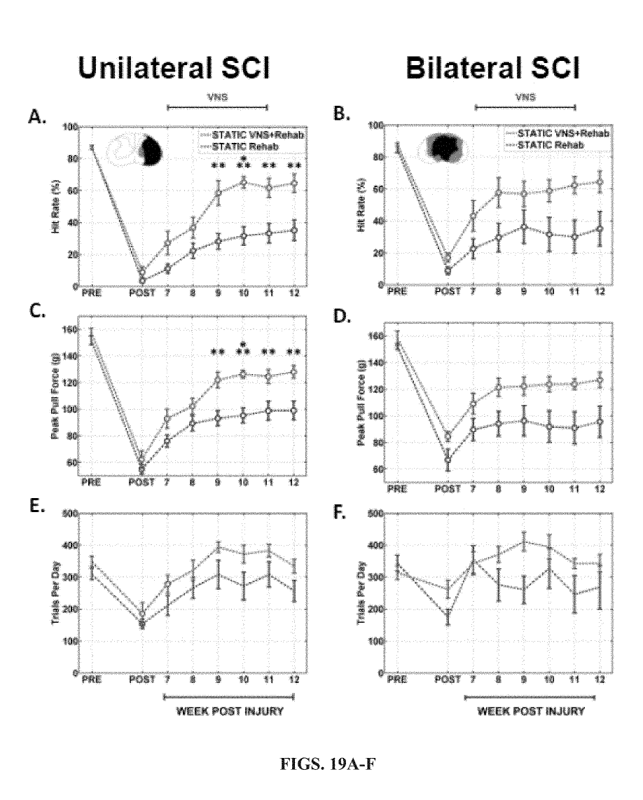

FIGS. 19A-F: VNS paired with rehabilitative training improved forelimb recovery as measured by the fixed 120 g threshold task. (FIGS. 19A-B) VNS paired with rehabilitative training (N=14) improved hit rate (>120 g pulls) compared to rehabilitative training without VNS (N=17) in rats with unilateral SCI. One day per week, rats with unilateral SCI (FIG. 19A) or bilateral SCI (FIG. 19B) were tested on the same static task that they were trained on prior to SCI. (FIGS. 19C-D) VNS paired with rehabilitative training improved force production compared to rehabilitative training without VNS even when the threshold for receiving a pellet (and VNS) was fixed. Significant group differences based on a two-way repeated measures ANOVA followed by independent sample t-tests are indicated by one asterisk for P<0.05, two asterisks for P<0.01, and three asterisks for P<0.001. Significant reductions compared to pre-lesion performance based on a two-way repeated measures ANOVA followed by simple contrasts are indicated by open symbols (P<0.05). VNS+Rehab rats received significantly more pellets then Rehab rats on weeks 9 through 12 (p<0.05),

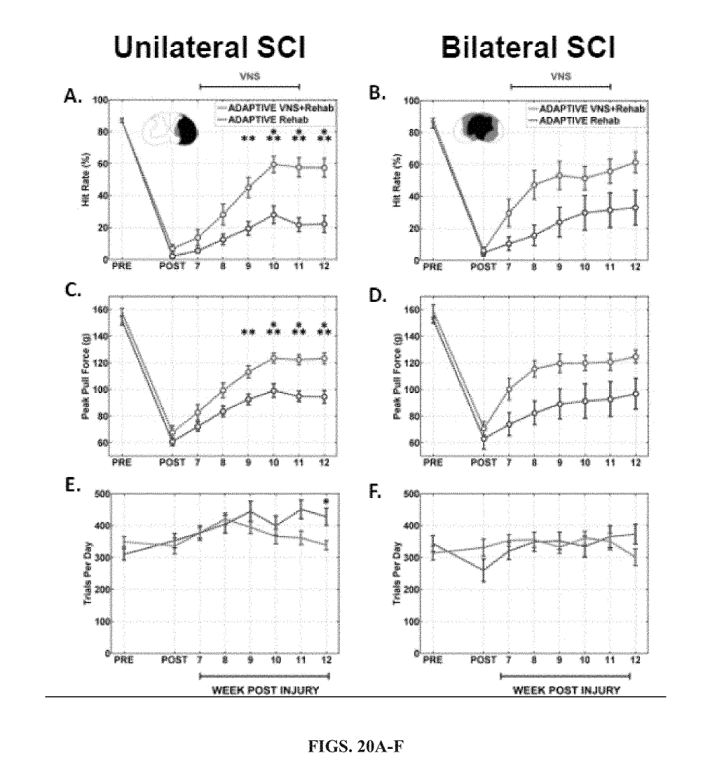

FIGS. 20A-F: VNS paired with rehabilitative training improved forelimb recovery as measured by the adaptive threshold task. (FIGS. 20A&B) VNS paired with rehabilitative training (N=14) improved hit rate (>120 g pulls) compared to rehabilitative training without VNS (N=17) in rats with unilateral SCI. Four out of five days of training, rats with unilateral SCI (FIG. 20A) or bilateral SCI (FIG. 20B) were tested on an adaptive threshold task that delivered a pellet (and VNS) on any trial that exceeded the median of the last 10 trials. (FIGS. 20C-D) VNS paired with rehabilitative training improved force production compared to rehabilitative training without VNS. Significant group differences based on a two-way repeated measures ANOVA followed by independent sample t-tests are indicated by one asterisk for P<0.05, two asterisks for P<0.01, and three asterisks for P<0.001. Significant reductions compared to pre-lesion performance based on a two-way repeated measures ANOVA followed by simple contrasts are indicated by open symbols (P<0.05). The fixed threshold task caused rats to pull slightly harder (3 g) than the adaptive threshold task (p<0.05). The fixed threshold task caused rats to initiate 20% fewer trials than the adaptive threshold task (p<0.05), presumably because the task was harder and yielded fewer rewards, VNS+Rehab rats received approximately the same number of food pellets as Rehab rats each week (p>0.5).

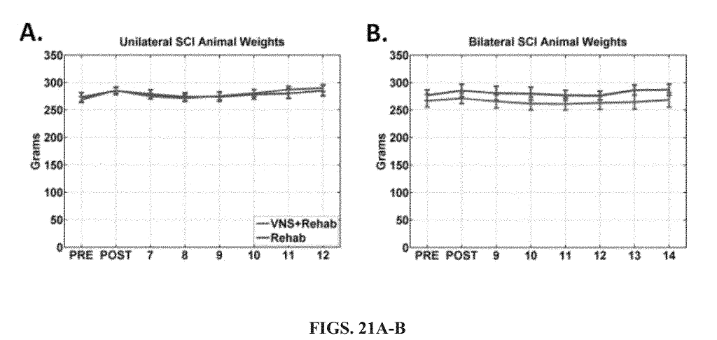

FIGS. 21A-B: VNS paired with rehabilitative training did not alter animal weights for unilateral or bilateral SCI rats. Animal weights for the unilateral (FIG. 21A) and. bilateral (FIG. 21B) SCI studies. There were no significant differences across time or group. VNS did not alter animal weight, which suggests that VNS did not improve forelimb motor performance (FIG. 1) by altering animal size. Results are from two-way repeated measure ANOVAs (unilateral SCI: VNS+Rehab, N=14; Rehab, N=17; bilateral SCI: VNS+Rehab, N=8; Rehab, N=8).

### DESCRIPTION OF ILLUSTRATIVE EMBODIMENTS

The impairments that result from spinal cord injury (SCA) are primarily determined by the location and extent of the damage. It has long been assumed that the degree of functional recovery is similarly determined by the lesion, but there is growing evidence that this assumption is often incorrect. The studies reported here tested whether functional recovery following incomplete SCI is primarily limited by insufficient or ineffective neural plasticity. Repeatedly pairing brief bursts of vagus nerve stimulation (VNS) with forelimb rehabilitation beginning six weeks after cervical SCI in rats generated therapeutic plasticity and promoted 77% more recovery of forelimb function compared to intense rehabilitation alone. The addition of VNS as an adjuvant to rehabilitation substantially improved the anatomical and physiological connectivity of motor circuits, without altering the extent of spinal cord damage. The finding that neural plasticity, and not lesion extent, primarily limits recovery from SCI provides new hope for patients and suggests that plasticity-based therapies may prove to be clinically useful. Thus, the present disclosure provides methods for the treatment of SCI in subjects by administering VNS, particularly in combination with rehabilitation.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term “spinal cord injury” (SCI) means any microscopic or macroscopic injury, wound, or damage to the spinal cord. Spinal cord injury may be an acquired injury to the spinal cord caused by an external physical force or as the result of a medical condition. Methods for diagnosing spinal cord injury are well-established in the art. Causes of spinal cord injury may include trauma (e.g., by motor vehicle accident, gunshot, or falls), or disease (polio, spina bifida, or Friedreich's Ataxia). Spinal cord injury may be an injury in which the spinal cord is partially or fully severed. Examples of spinal cord injuries in which the spinal cord is not severed may include contusion/bruising or partial transection of the spinal cord. Spinal cord injury may, in certain embodiments, include injuries in which the spinal cord is not severed. SCI includes injuries that occur at various points along the spine, e.g., at or below any of the eight cervical vertebrae or the twelve thoracic vertebrae or at L-1 or L-2. Spinal cord injury may also include trauma resulting from surgery, radiation, or other medical procedures.

As used herein, the term “lesion” refers to any pathological or traumatic discontinuity of tissue or loss of function of a part thereof. For example, lesions includes any injury associated with the spinal cord, for example, but not limited to contusions, compression injuries, etc.

The terms “administer”, “administering”, “administration”, and the like, as used herein, refer to the methods that are used to enable delivery of agents or compositions to the desired site of biological action. In particular embodiments, administering refers to the delivery of an electrical impulse to the vagus nerve.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

The terms “treat”, “treating” or “treatment”, and other grammatical equivalents as used herein, include alleviating, inhibiting or reducing symptoms, reducing or inhibiting severity of, reducing incidence of, prophylactic treatment of reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

### II. METHODS OF TREATMENT

Embodiments of the present disclosure provides methods for treating an individual having a symptom of, a disease, a disorder, or a condition related to, a spinal cord injury, comprising administering to the individual a therapeutically effective amount of VNS. VNS is administered in an amount effective to ameliorate, eliminate or prevent one or more symptoms of spinal cord injury, such as the symptoms of primary or secondary spinal cord injury. As used herein, “one or more symptoms” includes objectively measurable parameters, such as degree of inflammation, immune response, gene expression within the site of injury that is correlated with the healing process, quality and extent of scarring at the site of injury, improvement in the patient's motor and sensory function, and subjectively measurable parameters, such as patient well-being, patient perception of improvement in motor and sensory function, perception of lessening of pain or discomfort associated with the SCI.

### A. Spinal Cord Injury

Spinal cord injury can be considered as taking two forms. As defined herein, the primary injury is the initial injury, caused for example by an accident or trauma. As defined herein, the secondary injury is damage which develops later, for example in the minutes, hours, days and months following the primary injury. In particular, the present methods can be used to treat the primary injury or to prevent or limit the extent of secondary injury after primary injury has occurred. In certain embodiments, the individual is an animal, preferably a mammal, more preferably a non-human primate. In certain embodiments, the individual is a human patient. The individual can be a male or female subject. In certain embodiments, the subject is a non-human animal, such as, for instance, a cow, sheep, goat, horse, dog, cat, rabbit, rat or mouse.

Secondary injury may occur as a result of compression or spinal instability. Secondary injury can result from, for example, cellular hypoxia, oligaemia and/or edema due to an injury-induced neurochemical cascade. All of these conditions may be exacerbated by hypotension. Secondary injury can also be due to entry of immune cells, which release free radicals, into the spinal cord. In addition, trauma can cause the release of excess neurotransmitters, leading to excitotoxicity or secondary damage from overexcited nerve cells. Cells may die after spinal cord injury either by necrosis or apoptosis. Axons may also be damaged and nerve cells in the spinal cord below the lesion may die.

SCI is an insult to the spinal cord resulting in a change, either temporary or permanent, in its normal motor, sensory, or autonomic function. SCI includes conditions known as tetraplegia (formerly known as quadriplegia) and paraplegia. Thus, in some embodiments of the methods of treatment of SCI provided herein, the individual having a symptom of, or a disease disorder, or condition related to, an SCI is tetraplegic or paraplegic.

Tetraplegia refers to injury to the spinal cord in the cervical region, characterized by impairment or loss of motor and/or sensory function in the cervical segments of the spinal cord due to damage of neural elements within the spinal canal. Tetraplegia results in impairment of function in the arms as well as in the trunk, legs and pelvic organs. It does not include brachial plexus lesions or injury to peripheral nerves outside the neural canal.

Paraplegia refers to impairment or loss of motor and/or sensory function in the thoracic, lumbar or sacral (but not cervical) segments of the spinal cord, secondary to damage of neural elements within the spinal canal. With paraplegia, arm functioning is spared, but, depending on the level of injury, the trunk, legs and pelvic organs may be involved. The term is used in referring to cauda equina and conus medullaris injuries, but not to lumbosacral plexus lesions or injury to peripheral nerves outside the neural canal.

Common causes of SCI include, but are not limited to, motor vehicle accidents, falls, violence, sports injuries, vascular disorders, tumors, infectious conditions, spondylosis, latrogenic injuries (especially after spinal injections and epidural catheter placement), vertebral fractures secondary to osteoporosis, and developmental disorders. In certain embodiments, the SCI can result from blunt force trauma, compression, or displacement. In certain embodiments, the spinal cord is completely severed. In certain other embodiments, the spinal cord is damaged, e.g., partially severed or cut, but not completely severed. In other embodiments, the spinal cord is compressed, e.g., through damage to the bony structure of the spinal column, displacement of one or more vertebrae relative to other vertebrae, inflammation or swelling of adjacent tissues, or the like.

The SCI may be at one or more of the cervical vertebrae, thoracic vertebrae, lumbar vertebrae, and/or sacral vertebrae. In certain embodiments, the SCI is at vertebra C1, C2, C3, C4, C5, C6 or C7; or at vertebra T1, T2, T3, T4, T5, T6, T7, T8, T9, T10, T11 or T12; or at vertebra L1, L2, L3, L4 or L5. In certain other embodiments, the SCI is to a spinal root exiting the spinal column between C1 and C2; between C2 and C3; Between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5. In certain embodiments, the injury is to the cervical cord, thoracic cord, or lumbrosacral cord. In some embodiments, the injury is to the cones, one or more nerves in the cauda equine, or at the occiput.

In certain embodiments, a symptom of an SCI is numbness in one or more dermatomes (i.e., a patch of skin innervated by a given spinal cord level). In specific embodiments, the symptom of an SCI is numbness in one or more of dermatomes C1, C2, C3, C4, C5, C6, C7, T2, T3, T4, T5, T6, T7, T8, T9, T10, T11, T12, L1, L2, L3, L4 or L5.

The methods of treating SCI provided herein also provide for the treatment of an individual having other classifications of SCI including, but not limited to, central cord syndrome, Brown-Séquard syndrome, anterior cord syndrome, colitis medullaris syndrome, and cauda equina syndrome.

Central cord syndrome often is associated with a cervical region injury and leads to greater weakness in the upper limbs than in the lower limbs, with sacral sensory sparing. Thus, in specific embodiments of the method of treating SCI, the therapeutically effective amount of VNS is an amount sufficient to cause a detectable improvement in one or more symptoms of central cord syndrome, including, but not limited to, greater weakness in the upper limbs than in the lower limbs, with sacral sensory sparing.

Brown-Séquard syndrome, which often is associated with a hemisection lesion of the cord, causes a relatively greater ipsilateral proprioceptive and motor loss, with contralateral loss of sensitivity to pain and temperature. Thus, in specific embodiments of the method of treating SCI, the therapeutically effective amount of VNS is an amount sufficient to cause a detectable improvement in one or more symptoms of Brown-Séquard syndrome, including, but not limited to, ipsilateral proprioceptive and motor loss, with contralateral loss of sensitivity to pain and temperature.

Anterior cord syndrome often is associated with a lesion causing variable loss of motor function and sensitivity to pain and temperature; proprioception is preserved. Thus, in specific embodiments of the method of treating SCI, the therapeutically effective amount of VNS is an amount sufficient to cause a detectable improvement in one or more symptoms of anterior cord syndrome, including, but not limited to, variable loss of motor function and sensitivity to pain and temperature.

Conus medullaris syndrome is associated with injury to the sacral cord and lumbar nerve roots leading to are bladder, bowel; and lower limbs; while the sacral segments occasionally may show preserved reflexes (e.g., bulbocavernosus and micturition reflexes). Thus, in specific embodiments of the method of treating SCI, the therapeutically effective amount of VNS is an amount sufficient to cause a detectable improvement in one or more symptoms of cones medullaris syndrome, including, but not limited to, are bladder, bowel, and lower limbs.

Cauda equina syndrome is due to injury to the lumbosacral nerve roots in the spinal canal, leading to are bladder; bowel; and lower limbs. Thus, in specific embodiments of the method of treating SCI, the therapeutically effective amount of VNS is an amount sufficient to cause a detectable improvement in one or more symptoms of cauda equina syndrome, including, but not limited to, are bladder, bowel, and lower limbs.

In some embodiments, an improvement in one or more symptoms of, or a reduction in the progression of one or more symptoms of SCI is detected in accordance with the International Standards for Neurological and. Functional Classification of Spinal Cord Injury. The International Standards for Neurological and Functional Classification of Spinal Cord Injury, published by the American Spinal Injury Association (ASIA), is a widely accepted system describing the level and extent of SCI based on a systematic motor and sensory examination of neurologic function (e.g., Marino et al., 2003; incorporated by reference in its entirety).

In particular embodiments, an improvement in one or more symptoms of, or a reduction in the progression of one or more symptoms of SCI is detected in accordance with the ASIA Impairment Scale (modified from the Frankel classification), using the following categories:
A—Complete: No sensory or motor function is preserved in sacral segments S4-S5.4 (“Complete” refers to the absence of sensory and motor functions in the lowest sacral segments).
B—Incomplete: Sensory, but not motor, function is preserved below the neurologic level and extends through sacral segments S4-S5. “Incomplete” refers to preservation of sensory or motor function below the level of injury, including the lowest sacral segments.
C—Incomplete: Motor function is preserved below the neurologic level, and most key muscles below the neurologic level have muscle grade less than 3.
D—Incomplete: Motor function is preserved below the neurologic level, and most key muscles below the neurologic level have muscle grade greater than or equal to 3.
E—Normal: Sensory and motor functions are normal.

Thus, in a specific embodiment of the method of treating SCI provided herein, the therapeutically effective amount of VNS is an amount sufficient to cause a decrease an impairment according to the ASIA impairment scale (AIS). In some embodiments, the decrease is a one, two, three, or four grade reduction in impairment, wherein one grade corresponds to a single category improvement, for example, a reduction in impairment from category D to category E. In some embodiments, the therapeutically effective amount of VNS is an amount sufficient to convert an individual classified as ASIA A to ASIA B, ASIA C, ASIA D or ASIA E according to the AIS. In some embodiments, the therapeutically effective amount of VNS is an amount sufficient to convert an individual classified as ASIA B to ASIA C, ASIA D or ASIA E according to the AIS. In some embodiments, the therapeutically effective amount of VNS is an amount sufficient to convert an individual classified as ASIA C to ASIA D or ASIA E according to the AIS. In some embodiments, the therapeutically effective amount VNS is an amount sufficient to convert an individual classified as ASIA D to ASIA E according to the AIS.

### B. Vagus Nerve Stimulation

The vagus nerve (i.e., the tenth cranial nerve, paired left and right) is composed of motor and sensory fibers. The vagus nerve leaves the cranium, passes down the neck within the carotid sheath to the root of the neck, then passes to the chest and abdomen, where it contributes to the innervation of the viscera. A vagus nerve in a human consists of over 100,000 nerve fibers (i.e., axons), mostly organized into groups. The groups are contained within fascicles of varying sizes, which branch and converge along the nerve. Under normal physiological conditions, each fiber conducts electrical impulses only in one direction, which is defined to be the orthodromic direction, and which is opposite the antidromic direction. However, external electrical stimulation of the nerve may produce action potentials that propagate in orthodromic and antidromic directions. Besides efferent output fibers that convey signals to the various organs in the body from the central nervous system, the vagus nerve conveys sensory (afferent) information about the state of the body's organs back to the central nervous system. Some 80-90% of the nerve fibers in the vagus nerve are afferent (sensory) nerves, communicating the state of the viscera to the central nervous system.

The largest nerve fibers within a left or right vagus nerve are approximately 20 μm in diameter and are heavily myelinated, whereas only the smallest nerve fibers of less than about 1 μm in diameter are completely unmyelinated. When the distal part of a nerve is electrically stimulated, a compound action potential may be recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories, with approximate diameters as follows: A-alpha fibers (afferent or efferent fibers, 12-20 μm diameter), A-beta fibers (afferent or efferent fibers, 5-12 μm). A-gamma fibers (efferent fibers, 3-7 μm). A-delta fibers (afferent fibers, 2-5 μm), B fibers (1-3 μm) and C fibers (unmyelinated, 0.4-1.2 μm). The diameters of group A and group B fibers include the thickness of the myelin sheaths.

The vagus (or vagal) afferent nerve fibers arise from cell bodies located in the vagal sensory ganglia. These ganglia take the form of swellings found in the cervical aspect of the vagus nerve just caudal to the skull. There are two such ganglia, termed the inferior and superior vagal ganglia. They are also called the nodose and jugular ganglia, respectively. The jugular (superior) ganglion is a small ganglion on the vagus nerve just as it passes through the jugular foramen at the base of the skull. The nodose (inferior) ganglion is a ganglion on the vagus nerve located in the height of the transverse process of the first cervical vertebra.

1. Devices for Vagus Nerve Stimulation

Selected nerve fibers are stimulated in different embodiments of methods that make use of the disclosed electrical stimulation devices, including stimulation of the vagus nerve at a location in the patient's neck. At that location, the vagus nerve is situated within the carotid sheath, near the carotid artery and the interior jugular vein. The carotid sheath is located at the lateral boundary of the retopharyngeal space on each side of the neck and deep to the sternocleidomastoid muscle. The left vagus nerve is sometimes selected for stimulation because stimulation of the right vagus nerve may produce undesired effects on the heart, but depending on the application, the right vagus nerve or both right and left vagus nerves may be stimulated instead.

Electrical stimulation of a nerve involves the direct depolarization of axons. When electrical current passes through an electrode placed in close proximity to a nerve, the axons are depolarized, and electrical signals travel along the nerve fibers. The intensity of stimulation will determine what portion of the axons are activated. A low-intensity stimulation will activate those axons that are most sensitive, i.e., those having the lowest threshold for the generation of action potentials. A more intense stimulus will activate a greater percentage of the axons.

Many such therapeutic applications of electrical stimulation involve the surgical implantation of electrodes within a patient. In contrast, devices may be used to stimulate nerves by transmitting energy to nerves and tissue non-invasively. The methods of VNS to treat SCI provided herein may comprise invasive (e.g., surgical implantation) or noninvasive (e.g., transcutaneous) devices. In particular aspects, noninvasive methods are used to administer VNS.

A medical procedure is defined as being non-invasive when no break in the skin (or other surface of the body, such as a wound bed) is created through use of the method, and when there is no contact with an internal body cavity beyond a body orifice (e.g., beyond the mouth or beyond the external auditory meatus of the ear). Such non-invasive procedures are distinguished from invasive procedures (including minimally invasive procedures) in that the invasive procedures insert a substance or device into or through the skin (or other surface of the body, such as a wound bed) or into an internal body cavity beyond a body orifice. For example, non-invasive stimulation of the cervical vagus nerve which involves stimulating specific afferent fibers of the vagus nerve to modulate brain function has been demonstrated in animal and human studies to treat a wide range of central nervous system disorders including headache (chronic and acute cluster and migraine), epilepsy, bronchoconstriction, anxiety disorders, depression, rhinitis, fibromyalgia, irritable bowel syndrome, PTSD, Alzheimer's disease, and autism.

In some embodiments, VNS is administered by transcutaneous electrical stimulation of a nerve which is non-invasive because it involves attaching electrodes to the skin, or otherwise stimulating at or beyond the surface of the skin or using a form-fitting conductive garment, without breaking the skin. In contrast, percutaneous electrical stimulation of a nerve is minimally invasive because it involves the introduction of an electrode under the skin, via needle-puncture of the skin. Another form of non-invasive electrical stimulation is magnetic stimulation. It involves the induction, by a time-varying magnetic field, of electrical fields and current within tissue; in accordance with Faraday's law of induction. Magnetic stimulation is non-invasive because the magnetic field is produced by passing a time-varying current through a coil positioned outside the body. An electric field is induced at a distance, causing electric current to flow within electrically conducting bodily tissue. The electrical circuits for magnetic stimulators are generally, complex and expensive and use a high current impulse generator that may produce discharge currents of 5,000 amps or more, which is passed through the stimulator coil to produce a magnetic pulse.

The methods of the present disclosure rely upon modulated electrical stimulation of the vagus nerve. Such electrical stimulation can be achieved by a variety of different methods known in the art. By way of example, such electrical stimulation can be achieved via the use of a. neurostimulating device which can be, but does not necessarily have to be, implanted within the subject's body. Forms of neurostimulating devices or accessories thereof that can be employed in the methods disclosed herein are described in U.S. Pat. Nos. 4,573,481; 4,702,254; 4,867,164; 4,920,979; 4,979,511; 5,025,807; 5,154,172; 5,179,950; 5,186,170; 5,215,089; 5,222,494; 5,235,980, 5,237,991; 5,251,634; 5,269,303; 5,304,206; and 5,351,394, and U.S. Patent Publication No. 2011/0276112. In particular aspects, the device is an implantable pulse generator, such as the Vivistim system produced by MicroTransponder, Inc.

In electrical stimulator device may be applied to the patient's neck. In a preferred embodiment, the stimulator comprises two electrodes that lie side-by-side within separate stimulator heads, wherein the electrodes are separated by electrically insulating material. Each electrode and the patient's skin are connected electrically through an electrically conducting medium that extends from the skin to the electrode. The level of stimulation power may be adjusted with a wheel or other control feature that also serves as an on/off switch.

The neurostimulator can utilize a conventional microprocessor and other standard electrical and electronic components, and in the case of an implanted device, communicates with a programmer and/or monitor located externally to the subject's body by asynchronous serial communication for controlling or indicating states of the device. Passwords, handshakes, and parity checks can be employed for data integrity. The neurostimulator also includes means for conserving energy, which is important in any battery operated device, and especially where the device is implanted for medical treatment, and means for providing various safety functions, such as preventing accidental reset of the device.

The stimulus generator can be implanted in the patient's body in a pocket formed by the surgeon just below the skin in the chest in much the same manner as a cardiac pacemaker would be implanted, although a primarily external neurostimulator can also be employed. The neurostimulator also includes implantable stimulating electrodes, together with a lead system for applying the output signal of the stimulus generator to the patient's vagus nerve. Components external to the patient's body include a programming wand for telemetry of parameter changes to the stimulus generator and monitoring signals from the generator, and a computer and associated software for adjustment of parameters and control of communication between the generator, the programming wand, and the computer. A stimulating nerve electrode set is conductively connected to the distal end of an insulated electrically conductive lead assembly attached at its proximal end to a connector. The electrode set can be a bipolar stimulating electrode of the type described in U.S. Pat. No. 4,573,481. The electrode assembly is surgically implanted on the vagus nerve in the patient's neck. The two electrodes are wrapped about the vagus nerve, and the assembly can be secured to the nerve by a spiral anchoring tether such as that disclosed in U.S. Pat. No. 4,979,511. The leaks) is(are) secured, while retaining the ability to flex with movement of the chest and neck, by a suture connection to nearby tissue.

In conjunction with its microprocessor-based logic and control circuitry, the stimulus generator can include a battery or set of batteries which can be of any reliable, long-lasting type conventionally employed for powering implantable medical electronic devices, such as those employed in implantable cardiac pacemakers or defibrillators. For example, the battery can be a single lithium thionyl chloride cell. The terminals of the cell are connected to the input side of a voltage regulator which smooths the battery output to produce a clean, steady output voltage, and provides enhancement thereof such as voltage multiplication or division if required.

The voltage regulator supplies power to the logic and control section, which includes a microprocessor and controls the programmable functions of the device. Among these programmable functions are output current, output signal frequency, output signal pulse width, output signal on-time, output signal off-time, daily treatment time for continuous or periodic modulation of vagal activity, and output signal-start delay time. Such programmability allows the output signal to be selectively crafted for application to the stimulating electrode set to obtain the desired modulation of vagal activity. Timing signals for the logic and control functions of the generator are provided by a crystal oscillator.

A built-in antenna enables communication between the implanted stimulus generator and the external electronics, including both programming and monitoring devices, to permit the device to receive programming signals for parameter changes, and to transmit telemetry information from and to the programming wand. Once the system is programmed, it can operate continuously at the programmed settings until they are reprogrammed by means of the external computer and the programming wand.

The logic and control section of the stimulus generator controls an output circuit or section which generates the programmed signal levels appropriate for the condition being treated. The output section and its programmed output signal are coupled (e.g., directly, capacitively, or inductively) to an electrical connector on the housing of the generator and to a lead assembly connected to the stimulating electrodes. Thus, the programmed output signal of the stimulus generator can be applied to the electrode set implanted on the subject's vagus nerve to modulate vagal activity in the desired manner.

The housing in which the stimulus generator is encased is hermetically sealed and composed of a material such as titanium, which is biologically compatible with the fluids and tissues of the subject's body.

The stimulus generator can be programmed using a personal computer employing appropriate software and a programming wand. The wand and software permit non-invasive communication with the generator after the latter is implanted, which is useful for both activation and monitoring functions. Programming capabilities should include the ability to modify the adjustable parameters of the stimulus generator and its output signal, to test device diagnostics, and to store and retrieve telemetered data.

Diagnostics testing should be implemented to verify proper operation of the device. The nerve electrodes are capable of indefinite use absent indication of a problem with them observed on such testing.

2. Parameters for Vagus Nerve Stimulation

A source of power supplies a pulse of electric charge to the electrodes, such that the electrodes produce an electric current and/or an electric field within the patient. The electrical stimulator is configured to induce a peak pulse voltage sufficient to produce an electric field in the vicinity of a nerve such as a vagus nerve, to cause the nerve to depolarize and reach a threshold for action potential propagation. By way of example, the threshold electric field for stimulation of the nerve may be about 8 V/m at 1000 Hz. For example, the device may produce an electric field within the patient of about 10 to 600 V/m (preferably less than 100 V/m) and an electrical field gradient of greater than 2 V/m/mm. Electric fields that are produced at the vagus nerve are generally sufficient to excite all myelinated A and B fibers, but not necessarily the unmyelinated C fibers. However, by using a reduced amplitude of stimulation, excitation of A-delta and B fibers may also be avoided,

Current passing through an electrode may be about 0 to 40 mA, with voltage across the electrodes of about 0 to 30 volts. The current is passed through the electrodes in bursts of pulses. There may be 1 to 30 pulses per burst, such as 5, 10, 11, 12, 13, 14, 15, 16, 18, 19, 20 or 25 pulses per burst, particularly 15 or 16 pulses per burst. Each pulse within a burst has a duration of about 20 to 1000 microseconds, such as 30, 40, 50, 60, 70, 80, 90, 100, 125, 150, 175, or 200 microseconds, preferably 100 microseconds. A burst followed by a silent inter-burst interval repeats at 1 to 5000 bursts per second (bps, similar to Hz), preferably at 15-50 bps, and even more preferably at 25 bps. The preferred shape of each pulse is a full sinusoidal wave. In certain embodiments, the electrical signal is applied one to 150 times during a therapy session, such as 10, 25, 50, 75, or 100 times during a VNS treatment. The vagus nerve stimulation treatment according may be conducted for thirty seconds to five minutes, preferably about 90 seconds to about three minutes and more preferably about two minutes (each defined as a single dose).

The electric pulse train of the VNS may have a current amplitude of 0.1 to 2.0 milliamps (mA), such as between 0.4 to 1.0 mA, or between 0.7 to 0.9 mA, such as at around 0.8 mA. The electric pulse train may also have a duration of 30 to 5000 milliseconds (ms), such as 125 to 2000 ms, 400 to 600 ms, or 500 ms. For example, the electric pulse train with a duration of 500 ms typically consists of 15 pulses at 30 hz. An increase in pulse train duration would be associated with an increase in the number of pulses or a decrease in frequency. Conversely, a decrease in pulse train duration would be associated with a decrease in the number of pulses or an increase in frequency.

In some embodiments, the VNS may be applied continuously for a given period of time. The term “continuously stimulate” as defined herein means stimulation that follows a certain On/Off pattern continuously 24 hours/day. For example, existing implantable vagal nerve stimulators “continuously stimulate” the vagus nerve with a pattern of 30 seconds ON/5 minutes OFF for 24 hours/day and seven days/week. However, the treatment may then be modified on an individualized basis, depending on the response of each particular patient.

The VNS can be administered 1 day to 6 months, up to years after injury. For example, the individual can be treated immediately after injury, or within 1, 2, 3, 4, 5, 6 days of injury, or within 1, 2, 3, 4, 5, 6,7, 9, 10, 11, 12, 13, 13, 15, 16, 17, 18, 19, 20, 25, 30, 35, 40, 45, 50 days or more of injury, or within 1, 2, 3, 4, 5, 6, 7, 8, 9, 10 or more years after injury.

The preferred stimulator shapes an elongated electric field of effect that can be oriented parallel to a long nerve, such as a vagus. By selecting a suitable waveform to stimulate the nerve, along with suitable parameters such as current, voltage, pulse width, pulses per burst, or inter-burst interval, the stimulator produces a correspondingly selective physiological response in an individual patient. Such a suitable waveform and parameters are simultaneously selected to avoid substantially stimulating nerves and tissue other than the target nerve, particularly avoiding the stimulation of nerves in the skin that produce pain.

The methods for verifying and monitoring stimulation of the vagus nerve rely on the stimulated vagus nerve causing some physiological response that can be measured, such as some change in the patient's voice (by virtue of stimulation of a recurrent laryngeal nerve, which is a branch of the vagus nerve), autonomic nervous system, evoked potential, chemistry of the blood, or blood flow within the brain are described, for example, in U.S. Pat. No. 9,254,383.

### C. Combination Therapies

The methods for treating SCI provided herein further encompass treating SCI by administering a therapeutically effective amount of VNS in conjunction with one or more therapies or treatments used in the course of treating SCI. The one or more additional therapies may be used prior to, concurrent with, or after administration of the VNS.

In particular embodiments, patients undergo conventional rehabilitation through physical therapy, such as repetitive voluntary movement training and/or strength training, in combination with VNS for the treatment of SCI. The VNS may be administered before, during, or after each rehabilitation session. In particular, the VNS is administered 1 to 150 times during each rehabilitation session.

In some embodiments, the one or more additional therapies comprise the application of therapeutic spinal traction. Therapeutic spinal traction uses manually or mechanically created forces to stretch and mobilize the spine, based on the application of a force (usually a weight) along the longitudinal axis of the spinal column. If the neck or cervical segments are fractured, traction may straighten out and decompress the vertebral column.

In other embodiments, the one or more additional therapies comprise surgical stabilization of the spine, e.g. through the insertion of rods and screws to properly align the vertebral column or fuse adjacent vertebrae to strengthen the vertebra, promote bone re-growth, and reduce the likelihood of further SCI in the future.

Additional therapeutic agents can include corticosteroids, anticoagulants (e.g., heparin), and neuroprotective agents (e.g., methylprednisolone sodium succinate (MPSS), GM-1 (Sygen), Gacylidine (GK-11), thyrotropin releasing hormone, monocycline (minocycline), lithium or erythropoietin (EPO)). In other embodiments the therapeutic agent is inosine, rolipram, ATI-355 (NOGO), chondroitinase, fampridine (4-aminopyrideine), Gabapentin, or a Rho antagonist (e.g., Cethrin®). In another embodiment, the therapeutic agent is an immunomodulatory or immunosuppressive agent, e.g., Cyclosporin A, FTY506 (tacrolimus) or FTY720. In other embodiments, the therapeutic agent is a population of cells such as autologous macrophages, bone marrow stromal cells, nasal olfactory ensheathing cells, embryonic olfactory cortex cells, or Schwann cells.

Further examples of a pharmacological therapeutic agents that may be used in the present methods include an anti-inflammatory agent. Anti-inflammatory agents include, but are not limited to non-steroidal anti-inflammatory agents (e.g., naproxen, ibuprofen, celeocobix) and steroidal anti-inflammatory agents (e.g., glucocorticoids, dexamethasone, methylprednisolone). Other agents that can be used in combination with VNS can include, but are not limited to antioxidants, calcium blockers, drugs that control excitotoxicity, and drugs that enhance axon signaling, such as 4-aminopyridine. Still further other agents that can be used in combination with VNS may also include agents designed to promote regeneration by using trophic factors, and growth-inhibiting substances. Yet further, non-pharmacological interventions may also be used in combination with VNS, such as transplantation, peripheral nerve grafts, hypothermia (cooling).

Additional therapies can include neuroregenerative agents, neuroprotective agents, neurotrophic factors, growth factors, cytokines, chemokines, antibodies, inhibitors, antibiotics, immunosuppressive agents, steroids, anti-fungals, anti-virals or other cell types. In even more particular embodiments, the neuroprotective agent is for example dopamine D3 receptor agonists, the neurotrophic factors are for example BDNF, NT-3, NT-4, CNTF, NGF, or GDNF; the antibodies are for example IN-I anti-NOGO antibodies; the inhibitor is for example the PDE4 inhibitor rollipram; the immunosuppressive agents are for example corticosteroids, cyclosporine, tacrolimus, sirolimus, methotrexate, azathiopine, mercatopurine, cytotoxic antibiotics, polyclonal and monoclonal antibodies such as anti-T-cell receptor (CD23) and anti-IL2 receptor (CD25) antibodies, interferon, opioids, TNF binding proteins, mycophenolate, and small biological agents such as FTY720; the antibiotics are pikromycin, narbomycin, methymycin, neomethymycin; the steroid is methylprednisolone; and the cell types are for example differentiated AMP cells, or a mixture of differentiated and undifferentiated AMP cells, or a mixture of AMP cells (differentiated and/or undifferentiated) and other cells such as neural stem cells or any other progenitor cell or cells that are treated in such a way as to augment the AMP cells or AMP cell activity. Examples of cells include stem cells, neuroprogenitor cells, embryonic stem cells, neural stem cells, mesenchymal stromal cells, Schwann cells, induced pluripotent stern cells, neurons or a combination thereof. In the presence of ROS, stem cells either do not survive or differentiate. These cells could be mixed with nano-SOD/catalase to enhance their survival and differentiation into neuronal cells. One could inject a combination of cells and nano-SOD/catalase to facilitate rapid repair of injured spinal cord. Examples of growth factors include brain derived neurotrophic factor (BDNF), neurotrophin-3 (NT-3), acidic fibroblast growth factor (aFGF; FGF-1), hepatocyte growth factor (HGF) or a combination thereof. Examples of additional antioxidants include glutathione peroxidase, glutathione reductase, caspase inhibitors, or a combination thereof. Examples of hormones include one or more thyroid hormones. In addition, vitamins such as C, E, A (beta-carotene); nutrients such as lutein, lycopene, vitamin B2, coenzyme Q10; amino acids such as cysteine and herbs such as bilberry, turmeric (curcumin), grape seed or pine bark extracts and ginko can be used.

### III. EXAMPLE

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should; in light of the present disclosure, appreciate that many changes can be made in the specific embodiments which are disclosed and still obtain a like or similar result without departing from the spirit and scope of the disclosure.

### Example 1

### Effects of Vagus Nerve Stimulation

To determine the effect of vagus nerve stimulation (VNS), thirty-one rats were trained to reach through a narrow slit, grasp; and pull a handle with at least 120 grams of force (FIG. 1A PRE, FIG. 4A-B). After training, each rat received a contusion to the right spinal cord at C5/C6 (FIG. 5) and a cuff electrode on the left vagus nerve. After recovery, rats returned to the task and received twice daily rehabilitative training for seven weeks, After the first week of rehabilitation, half of the rats were randomized to receive a brief burst of VNS with each successful trial (FIG. 4C).

Unilateral spinal cord injury (SCI) reduced the hit rate on the isometric pull task by 94±1% and reduced average force production by 59±1% (FIGS. 1A & 1C, POST). As expected from previous studies, seven weeks of intensive daily rehabilitative training improved hit rate and force production; however, rats continued to exhibit a substantial impairment in forelimb function compared to pre-lesion performance (Rehab: FIGS. 1A & 1C) (Khodaparast et at., 2013; Hays et al., 2014; Pruitt et al., 2015). The addition of brief bursts of VNS delivered on successful trials during rehabilitative training significantly enhanced recovery compared to rehabilitative training without VNS (Rehab+VNS: FIG. 1A). With VNS, rats recovered to 67±9% of pre-lesion levels compared to 29±6% recovery without VNS (week 11: unpaired t-test, P=0.0002). Enhanced recovery was maintained after the cessation of VNS (week 12: FIG. 1A). Volitional forelimb strength recovered to a greater extent in rats that received VNS and the benefit persisted long after the end of VNS (FIG. 1C, Two-way repeated measures ANOVA, F[7,196]=160.4, P=6.6×10⁻³⁸). These results demonstrate that VNS can improve recovery from SCI.

Since bilateral damage to the cervical spinal cord is the most common SCI in humans and bilateral damage could limit both plasticity and recovery, the functional deficit and recovery was also quantified from midline cervical contusion in fifteen rats. The bilateral spinal cord lesions caused twice as much tissue damage (FIG. 6) and more than doubled the time to regain ambulation compared to unilateral SCI (FIG. 7). VNS paired with rehabilitative training significantly enhanced recovery from bilateral SCI compared to rehabilitative training without VNS (FIG. 1D, Two-way ANOVA, F[1,144]=40, P=5×10⁻⁹). Enhanced recovery was maintained after the cessation of VNS (week 14). This is the first demonstration that VNS can improve recovery from bilateral damage to the central nervous system and suggests that VNS-based therapies may prove to be clinically useful.

Enhanced behavioral recovery is consistent with the hypothesis that VNS paired with rehab may drive therapeutic neural plasticity, however enhanced improvement is insufficient to demonstrate neural plasticity. It was possible that VNS enhances recovery after spinal cord injury through some mechanism other than neural plasticity, such as a reduction in lesion size, muscle atrophy, pain or spasticity.

Additional behavioral testing, histology (FIGS. 5-6), awake behaving electrophysiology (FIG. 8), transsynaptic labeling (FIG. 2), and intracortical microstimulation (FIG. 3) studies were conducted to clarify the biological mechanism responsible for enhanced recovery following SCI. Unilateral SCI was used for these studies, because it is the most commonly used preclinical model of SCI and resulted in a lower mortality rate compared to bilateral contusion (15% vs. 30%).

VNS did not alter gray matter damage, white matter damage, or the anterior-posterior extent of SCI (FIGS. 5-6). Biceps EMG amplitude during volitional movement was not significantly different across the groups at any time (FIG. 9, Biceps EMG: F[2,12]=0.9, P=0.416;) (Ganzer et al., 2016). Forepaw sensitivity to thermal and tactile stimulation was not different across the groups at any time (FIG. 10, Thermal: F[2,18]=0.3, P=0.745; Tactile: F[2,44]=2.3, P=0.107). The EMG response to noxious thermal stimulation was elevated after SCI, which is consistent with earlier reports of post-SCI spasticity, but was not different across the groups at any time (FIG. 11; VNS+Rehab, PRE vs. Wk12: P=0.291; Rehab, PRE vs. Wk12: P=0.39). VNS did not alter the number of trials rats performed during rehabilitative training (FIGS. 1C & 1F). These results suggest that VNS did not enhance forelimb function by influencing motivation, lesion size, pain, muscle atrophy or spasticity.

Anatomical and physiological studies clearly demonstrate that VNS paired with rehabilitation increases neural plasticity compared to rehabilitation alone. Injection of pseudorabies virus (PRV-152) into the grasping muscles flexor digitorum profundus and palmaris longus was used to assay connectivity of descending motor circuits and resulted in transsynaptic labeling of neurons in layer 5 of motor cortex contralateral to the trained limb (FIG. 2A), SCI dramatically reduced the number of labeled cortical neurons in rats that received extensive rehab alone compared to naïve rats (FIG. 2B-D; 87±10% reduction, p<0.001). Rats that received VNS paired with rehabilitative training had substantially more motor cortex labeling compared to rats that received rehabilitative training without VNS (FIGS. 2B, 2D, 2E: 200±50% increase, p<0.001). VNS did not increase the proportion of primary motor neurons or spinal interneurons labeled by PRV (FIGS. 2C, 15), which suggests that much of the neural plasticity may have occurred above the level of the spinal cord. The improved anatomical connectivity of motor circuits when VNS is added to rehabilitation supports the hypothesis that even intense rehabilitation alone does not yield maximal recovery of motor system connectivity.

Intracortical microstimulation (ICMS) was used to confirm that VNS also improves the physiological connectivity of motor circuits (FIGS. 3A-C). Compared to rehab only rats, VNS±rehab rats had substantially more motor cortex sites that generated grasping movement of the digits (FIG. 3D; one-way MANOVA, F[2,19]=5, P=0.017; 1.6±0.3 mm² vs. 3.1±0.4 mm², P<0.05). The increased neural representation of grasping when VNS was added to rehabilitation suggests that weeks of even intensive rehabilitation fails to yield maximal neural plasticity.

To determine whether the beneficial effects of VNS can generalize to assessments other than the training conditions, grip strength was evaluated using an unskilled paradigm conducted in a different context (FIGS. 19A-F). VNS during rehab improved the impaired grip strength in rats with unilateral SCI compared to unilateral SCI rats that received rehab alone. Bilateral SCI did not reduce grip strength, which confirms that the two lesion types yield distinct deficits. These results indicate that performance on the isometric pull task does not simply reflect impaired grip strength and suggest that VNS paired with rehabilitative training improves voluntary motor control.

Thus, these findings provide the first direct demonstration that VNS paired with rehabilitative training can generate beneficial neural plasticity. The mechanisms through which VNS enhances SCI recovery are not yet fully understood. However, it is clear that delivery of VNS during rehabilitation can increase the number of functional synaptic connections in descending networks from the motor cortex to the target forelimb musculature (FIG. 18). Earlier studies suggest that the plasticity-enhancing property of VNS depends on the precise timing of VNS delivery during rehabilitation and the presence of an intact central cholinergic system. The observation that intensive rehabilitation is insufficient for optimal recovery from unilateral or bilateral SCI should support the search for new and clinically-viable methods to enhance neural plasticity.

The protocol used in this study and in earlier studies in stroke and tinnitus patients represents only 1% of the VNS protocol approved by the FDA for epilepsy and depression. A clinical trial evaluating VNS paired with rehabilitation in stroke patients indicates that the therapy is safe and can enhance rehabilitation. The absence of evidence of autonomic dysreflexia or other significant side effects in two rat models of SCI suggests that pairing rehabilitative training with VNS may also prove safe and effective in SCI patients. If VNS-directed plasticity is proven to be an effective adjuvant to rehabilitation of the motor symptoms of neurological disease, it may, be possible to develop new forms of VNS-enhanced rehabilitation to address sensory and cognitive symptoms.

### Example 2

### Materials and Methods

Subjects and Experimental Design. All procedures performed in the study were approved by the University of Texas at Dallas Institutional Animal Care and Use Committee. Adult female Sprague Dawley rats (N=58) used in this study were housed one per cage (12 hour light/dark cycle). A subset of these rats (N=9) received chronically implanted EMG electrodes into the long head of the biceps brachii of the trained forelimb to assess volitional and reflexive muscular dynamics. Rats were food deprived Monday-Friday (ad libitum access to water) and trained to proficiency on the isometric pull task using only the right forelimb. Rats were either subjected to a right side or midline cervical spinal contusion at spinal level C5/C6. Post-injury forelimb strength assessment occurred before and after headcap and nerve cuff implant surgery (see below). After cervical SCI, rats were placed into balanced treatment groups and received traditional rehabilitation or vagus nerve stimulation paired with rehabilitation. In a subset of rats, terminal motor cortex mapping or transsynaptic tracing experiments occurred the week following the end of therapy.

Volitional Forelimb Strength Assessment. All rats in the study were trained to proficiency on the isometric pull task similar to previous studies (Pruitt et al., 2014). The isometric pull task is an automated and quantitative means to measure multiple parameters of forelimb force generation (Sloan et al., 2015). Please refer to previous manuscripts for information on behavioral chamber dimensions, data acquisition software or animal training procedures (Ganzer et al., 2016).

After reaching task proficiency (85% of trials above 120 g), rats were given a unilateral or bilateral cervical SCI at C5/C6 (FIG. 4). Post-injury baseline strength assessment occurred during weeks 4 and 6 post-injury for unilateral SCI and during weeks 6 and 8 post-injury bilateral SCI rats. Post-injury baseline strength was used to create balanced treatment groups. Each post-injury strength assessment time point consisted of four 30 minute sessions across 2 consecutive days (again 2 thirty minute sessions per day) to assess forelimb strength (Day 1: 2 adaptive force threshold sessions (10 gram starting and 120 gram max threshold; adaptive threshold based on median of the previous 10 trials); Day 2: 1 static force threshold session (120 gram static threshold), and 1 adaptive force threshold session) similar to previous studies (Ganzer et al., 2016).

Therapy was then started following the last post-injury baseline assessment and continued for 6 weeks (FIG. 4). Each therapy week consisted of 5 days of training. Rats performed the task with an adaptive force threshold on days 1-4 and a static force threshold on day 5 of a given week.

Forelimb Withdrawal Assessment. Forelimb withdrawal to a thermal stimulus was performed similar to previous studies (Ganzer et al., 2016).

Forelimb Tactile Allodynia. Assessment. Rats were acclimated to suspended Plexiglas chambers (30 cm long×15 cm wide×20 cm high) with a wire mesh bottom (1 cm²) for 1 hour. Experimenters were blind to the group of the rat. Paw withdrawal (PW) thresholds are determined by applying von Frey filaments (4.31, 4.56, 4.74, 4.93, and 5.18) to the plantar aspect of the forepaws, and a response was indicated by a withdrawal of the paw. The withdrawal thresholds were determined by the Dixon up-down method. Maximum filament strengths were 15 g for the forepaws.

Forelimb Grip Assessment. Forelimb grip assessment was performed at POST and the final week of therapy for unilateral and bilateral SCI rats (FIG. 4). A group of uninjured rats proficient on the pull task were used for control (N=7). The grip assessment module consisted of 2 separate isometric bars attached to load cells for transducing grip force (FIG. 15). This allowed for simultaneous grip assessment for both forelimbs. Force transduction and measurement was made using a custom MATLAB interface. Rats were held at the hindquarters while horizontally suspended gripping each bar with all digits. Rats were then slowly pulled away from the module until grip broke similar to previous studies. Maximum grip values for uninjured control rats using our custom module were similar to other commercially available devices.

Surgeries and Vagus Nerve Stimulation. EMG, cervical SCI, VNS and transsynaptic tracing surgeries were performed using sterile technique under general anesthesia. Rats were anesthetized with ketamine (50 mg/kg), xylazine (20 mg/kg), and acepromazine (5 mg/kg) for all procedures. Heart rate and blood oxygenation was monitored during surgery. Antibiotic and analgesic treatments are listed below. All rats were given at least 7 days to recover from a given surgery before handling.

Chronic Electromyography (EMG) Implant Surgery. Prior to training on the isometric pull task (PRE, FIG. 4), a subset of rats (N=9) received chronically implanted intramuscular electrodes into the long head of the biceps brachii to monitor forelimb electromyography (EMG) similar to previous studies (Ganzer el al., 2016).

Cervical Spinal Cord Injury (cSCI) Surgery. After achieving isometric pull task proficiency, rats received either a right side (unilateral) or midline (bilateral) C5/C6 spinal cord contusion using surgical technique from previous studies (Ganzer et al., 2016). All rats were randomized post-injury into balanced treatment groups based on pull strength. Therefore, experimenters were blind to the group of the animal during surgery. A right side or bilateral dorsal C5 laminectomy was performed for rats receiving a unilateral or bilateral SCI, respectively. The vertebral column was stabilized using spinal microforceps. For unilateral SCI rats, the right spinal hemicord was rapidly contused using the Infinite Horizon Impact Device with a force of 200 kilodynes as previously reported (Precision Systems and Instrumentation, Lexington, Ky.; impactor tip diameter=1.25 mm) (Ganzer et al., 2016). For bilateral SCI rats, the midline of the spinal cord was rapidly contused with a force of 225 kilodynes (impactor tip diameter=2.5 mm). The skin overlying the exposed vertebrae was then closed in layers and the incised skin closed using surgical staples. All rats received Buprinex (s.c., 0.03 mg/kg, 1 day post-op), Baytril (s.c., 10 mg/kg, daily for 3 days) and Ringer's solution (s.c., 5 mL) following surgery and post-operatively if noted.

Animal health was monitored closely following SCI surgery. The time was documented for self-feeding and forelimb plantar placement during post-operative care and pain assessment. Details of post-injury recovery are reported in FIG. 7. Bilateral SCI rats took significantly longer to regain mobility and self-feeding (FIG, 7A; Recumbency) and forepaw plantar placement (FIG. 7B) compared to unilateral rats. Therefore, bilateral SCI rats started therapy 2 weeks later. All rats were monitored daily for 1 week post-injury. Midline SCI rats were hand fed twice daily and given Ringer's solution (s.c., 10 mL) for up to 1 week post-injury to maintain a healthy diet.

Vagus nerve stimulation cuff and headcap surgery. After the last post-injury baseline assessment, a two-channel connector headcap and vagus nerve stimulating cuff was implanted similar to previous studies (see Volitional Forelimb Strength Assessment section for post-injury assessment time points) (Khodaparast et al., 2013; Hays et al., 2014; Pruitt et al., 2015). Experimenters were blind to the treatment group of the animal. Stimulation of the left cervical branch of the vagus nerve was performed using low current levels to avoid cardiac effects. Incised skin was closed using suture. All rats received Baytril (s.c., 10 mg/kg) following surgery and as needed at the sign of infection. Heart rate and respiration were monitored during VNS cuff implant and the end of therapy to confirm VNS efficacy. No abberant alterations heart rate and respiration were observed during assessment ruling out autonomic dysregulation after injury.

Vagus nerve stimulation. VNS was automatically triggered by the behavioral software during performance of the isometric pull task: 15 pulse train at 30 Hz consisting of 100 μsec 0.8 mA biphasic pulses (Khodaparast et al., 2013; Hays et al., 2014; Pruitt et al., 2015).

Motor mapping surgery. Terminal mappings of motor cortex were performed during week 13 post-injury following the end of therapy. Rats were deeply anesthetized and a cisternal drain was performed to reduce ventricular pressure and cortical edema during mapping (Porter et al., 2012). A craniotomy was then performed to expose left motor cortex. Intracortical microstimulation (ICMS) was delivered in motor cortex at a depth of 1.75 mm using a low impedance tungsten microelectrode with an interpenetration resolution of 500 μm (100 kOhm-1 MOhm electrode impedance; FHC Inc., Bowdin, Md.; biphasic ICMS at 333 Hz, 50 ms duration, 200 μsec pulse duration, 0-200 μA current). Mapping experiments were performed double-blind with 2 experimenters. The first experimenter positioned the electrode for ICMS. The second experimenter was blind to the experimental group of the animal and electrode position, delivered ICMS and collected movement data. Movement threshold was first defined. ICMS current was then increased by 50% to facilitate movement classification using visual inspection. Movements were classified into the following categories similar to previous studies (Brown and. Teskey, 2014; Ganzer et al., 2016b): vibrissae, neck/jaw, digit, wrist, elbow, shoulder, hindlimb and trunk.

Transsynaptic tracing surgery. Transsynaptic tracer injections were performed in unilateral SCI rats during week 12 after injury under deep anesthesia with pseudorabies virus 152 (PRV-152; FIG. 4). PRV-152 was a generous gift from the lab of Dr. Lynn Enquist and colleagues at Princeton University and was grown using standard procedures. An incision was made over the medial face of the radius and ulna of the trained limb to expose the flexor digitorum profundus and palmaris longus (i.e. the forelimb grasping muscles). 15 μL of PRV-152 was injected into the belly of each muscle across three separate sites. The incision was then closed with non-absorbable suture. PRV-152 used in this study was ˜8.06±0.49×10⁸ plaque-forming units similar to previous studies (Gonzalez-Rothi et al., 2015). Rats were anesthetized with sodium pentobarbital (50 mg/kg, i.p.) and transcardially perfused with 4% paraformaldehyde in 0.1 M PBS (pH 7.5) at 6-6.5 days after injection. The brain and spinal cord were removed. Spinal roots were kept for anatomical reference. Tissue was then post-fixed overnight and cryoprotected in 30% sucrose.

Forelimb strength data acquisition. Custom software was used to display and record experimental data during the performance of the task similar to previous studies (Ganzer et al., 2016). A microcontroller board (Vulintus, Inc.) sampled the force transducer every 10 ms and relayed information to custom MATLAB software for offline analysis. For rats receiving VNS, stimulation was triggered by the behavioral software during isometric force threshold crossings.

EMG data acquisition. EMG data was recorded and conditioned during forelimb strength and hyperreflexia testing similar to previous studies (Ganzer et al., 2016). A trial was initiated when the rats exerted at least 10 grams of pull force or at the time of paw withdrawal from the thermal stimulus (FIGS. 4B, 8) (Ganzer et al., 2016). A. TTL pulse sent from the task microcontroller interface synchronized EMG signal recordings to the approximate time of each trial initiation.

Forelimb grip data acquisition. Custom software was used to display and record experimental data during forelimb grip assessment. A microcontroller board (Vulintus, Inc.) simultaneously sampled the 2 force transducers every 10 ms and relayed information to custom MATLAB software for offline analysis.

Data Analysis. All data are reported in text and figures as the mean±standard error of the mean. Statistical normality was assessed for all tests prior to analysis (SPSS; IBM). Regressions were performed using Pearson's Linear correlation (Graphpad Prism). An alpha of p<0.05 was considered significant for omnibus measures.

Forelimb strength data analyses. The hit rate (percent of trials>120 grams), peak force (maximum force generated in a trial), Force Energy (RMS of force profiles), Pull Speed (mean speed (grams/10 ms) were calculated in a trial of all rising phases of isometric force profiles) across rats for all assessment time points. When noted, static and adaptive sessions across time and group are assessed separately (for session details see Volitional Forelimb Strength Assessment section above). For unilateral and bilateral SCI studies, the effect of SCI and therapy on isometric pull task variables was assessed separately using two-way repeated measures ANOVAs for each group (VNS+Rehab and Rehab). The factor was Time with 8 levels (PRE, POST and the 6 weeks of therapy; see FIG. 4 for timeline). Differences across Time were assessed using Simple Contrasts (compared to PRE). Differences within a time point across group was assessed using Bonferroni corrected independent samples t-tests (alpha=0.05/number of comparisons) if needed.

Forelimb grip data analyses. Forelimb grip assessment was performed for unilateral and bilateral SCI rats at PRE, POST and the end of therapy. The effect of SCI and therapy on forelimb grip ability was assessed two-way ANOVAs for each group. Differences across Time were assessed using Simple Contrasts (compared to PRE). Differences were assessed using Tukey's post-hoc tests.

EMG & Pain data analyses. Biceps EMG activity was analyzed offline similar to previous studies (Ganzer et al., 2016). Peri-event time histograms (PETH) based analysis was performed for EMG during the isometric pull task (event=pull trial initiation) and noxious heat withdrawal testing (event=forepaw withdrawal). EMG PETH's were generated similar to previous studies. We calculate and report the EMG response magnitude (Ganzer et al., 2016). The effect of SCI and therapy on EMG response magnitude was assessed two-way repeated measures ANOVAs for each group. Differences across Time were assessed using Simple Contrasts (compared to PRE). Differences within a time point across group was assessed using Bonferroni corrected independent samples t-tests (alpha=0.05/number of comparisons) if needed.

Similarly, the Paw Withdrawal Threshold (g; tactile) and Latency (s; thermal) were calculated. Differences across Time and group were assessed as noted above.

Motor cortex mapping data analyses. The cortical area (mm²) and movement threshold (μA) was calculated for ICMS movements for each group. Movement area and threshold was assessed using two-way ANOVAs. The two factors were group with 3 levels (Naive, VNS+Rehab and Rehab) and movement type with 8 levels (vibrissae, neck/jaw; digit, wrist, elbow, shoulder, hindlimb and trunk). Differences were assessed using Tukey's post-hoc tests.

Transsynaptic tracing data analyses. PRV152 positive neuron counts were performed similar to previous studies assisted by custom software (Bareyre et al.,2004). Neuron counts were performed for the sensorimotor cortex using electrophysiological mapping boundaries and standard anatomical atlas reference (Paxinos and Watson, 2007). Sensorimotor cortex neuron counts were normalized within rats to the number of positively labeled putative motor neurons in the lower cervical spinal cord to derive relative neuron counts. PRV back-labeled putative motor neuron counts were performed similar to previous studies (Gonzalez et. al., 2015). Sensorimotor cortex and putative motor neuron counts and were analyzed using one-way ANOVAs. The factor was group with 3 levels (Naïve; VNS+Rehab and Rehab). Differences were assessed using Tukey's post-hoc tests.

SCI histological analyses. Spinal cord tissue was perfused, stained for Nissl and myelin and imaged similar to previous studies (Ganzer et al., 2016). cSCI lesion metrics were quantified using Image J software. For unilateral SCI rats, the rostral and caudal extent of spinal gray and white matter damage was expressed as the percentage of spared gray and white matter of the right hemicord with respect to the left hemicord. For bilateral SCI rats, the rostral and caudal extent of spinal damage was expressed as the percentage of spared gray and white matter for each hemicord with respect to a unilesioned rostral and caudal tissue reference within animals. Smallest and largest lesion outlines were fitted to a cartoon of spinal level C6 (FIGS. 5-6).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Bareyre et al., Nat Neurosci. 7(3):269-77, 2004.
Brown and Teskey, J Neurosci. 34(41):13574-85, 2014.
Dietz and Fouad, Brain, 137(3):654-67, 2014.
Ganzer et al., Behav Brain Res. 307:100-11, 2016.
Ganzer et al., Exp Neurol. S0014-4886(16)30021-8, 2016b.
Gonzalez-Rothi et al., J Neurotrauma. 32(12):893-907, 2015.
Harvery et al., Spinal Cord. 47(3):184-95, 2009.
Hays el al., Stroke, 45(10):3097-100, 2014.
Khodaparast et al., Neurobiology of disease, 60:80-8, 2013.
Marino et al., J. Spinal Cord Med. 26 Suppl 1:S50-6, 2003.
Paxinos and Watson, The Rat Brain in Stereotaxic Coordinates, Ed 6, Academic/Elsevier, Boston, 2007.
Perlmutter and Mink, Annu. Rev. Neurosci 29:229-257, 2006.
Porter et al., Cerebral cortex, 22(10):2365-74, 2012.
Pruitt et al., Brain Res. 1582:91-8, 2014.
Pruitt et al., Journal of Neurotrauma, 2015.
Sloan et al., PLoS One. 10(10):e0141254, 2015.
U.S. Pat. No. 4,573,481
U.S. Pat. No. 4,573,481
U.S. Pat. No. 4,702,254
U.S. Pat. No. 4,867,164
U.S. Pat. No. 4,920,979
U.S. Pat. No. 4,979,511
U.S. Pat. No. 4,979,511
U.S. Pat. No. 5,025,807
U.S. Pat. No. 5,154,172
U.S. Pat. No. 5,179,950
U.S. Pat. No. 5,186,170
U.S. Pat. No. 5,215,089
U.S. Pat. No. 5,222,494
U.S. Pat. No. 5,235,980
U.S. Pat. No. 5,237,991
U.S. Pat. No. 5,251,634
U.S. Pat. No. 5,269,303
U.S. Pat. No. 5,304,206
U.S. Pat. No. 5,351,394
U.S. Pat. No. 6,871,099
U.S. Pat. No. 9,254,383
U.S. Patent Publication Na. 2011/0276112

1. A method of treating a spinal cord injury in a subject comprising applying an electrical signal to a vagus nerve of said subject.

2. The method of claim 1, wherein treating results in increased in neural plasticity, increased motor circuit connectivity, improved motor function, improved sensory function, enhanced voluntary motor control, and/or prevention of secondary injury and/or wherein treating results in at least a 50% improvement in motor function.

3. (canceled)

4. The method of claim 1, wherein the electrical signal is administered in combination with rehabilitation.

5. The method of claim 1, wherein the electrical signal is administered concurrent with rehabilitation.

6. The method of claim 1, wherein the rehabilitation comprises physical therapy.

7. The method of claim 1, wherein the electrical signal is administered 1 day to 10 years after the spinal cord injury.

8. The method of claim 1, wherein the spinal cord injury is caused by contusion of the spinal cord, bruising of the spinal cord, loss of blood to the spinal cord, pressure on the spinal cord, cut spinal cord, severed spinal cord or is the result of a physical trauma, infection, insufficient blood flow, or a tumor.

9. (canceled)

10. The method of claim 1, wherein the spinal cord injury is complete spinal cord injury or incomplete spinal cord injury.

11. (canceled)

12. The method of claim 1, wherein the spinal cord injury is at one or more of the cervical vertebrae, thoracic vertebrae, lumbar vertebrae, or sacral vertebrae.

13. The method of claim 1, wherein the electrical signal is monophasic, biphasic, or a combination thereof.

14. (canceled)

15. The method of claim 1, wherein applying is further defined as transmitting said electrical signal transcutaneously to the subject to generate an electrical impulse at or near the vagus nerve fibers.

16-20. (canceled)

21. The method of claim 1, wherein the electrical signal comprises bursts of pulses with a frequency of 1 to 100 bursts per second.

22-23. (canceled)

24. The method of claim 21, wherein the pulses are full sinusoidal waves or square waves.

25. The method of claim 21, wherein each burst has a wave frequency of 1 to 100 Hz.

26. (canceled)

27. The method of claim 21, wherein each pulse is 10 to 1000 microseconds in duration.

28-29. (canceled)

30. The method of claim 21, wherein the electrical signal has a current of 0.1 to 2.0 mA.

31. (canceled)

32. The method of claim 21, wherein the electrical signal has a duration of 100 to 1000 milliseconds.

33. (canceled)

34. The method of claim 1, wherein the electrical signal is applied one to 500 times during a therapy session.

35. (canceled)

36. The method of claim 1, wherein the subject is human.

37. The method claim 1, further comprising administering at least one additional therapy.

38-43. (canceled)
